(19) United States
(12) Patent Application Publication
(10) Pub. No.: US 2003/0166192 A1
(43) Pub. Date: Sep. 4, 2003

(54) Inhibition of interleukin-1-beta secretion by card proteins

(76) Inventor: Emad S. Alnemri, Ambler, PA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/050,054

(22) Filed: Jan. 16, 2002

Related U.S. Application Data
(60) Provisional application No. 60/340,161, filed on Dec. 14, 2001.
(60) Provisional application No. 60/262,477, filed on Jan. 16, 2001.

(51) Int. Cl. C12N 9/99; C07H 21/04; C12P 21/02; C12N 5/06

(52) U.S. Cl. 435/184; 435/69.2; 435/320.1; 435/325; 536/23.2

(57) ABSTRACT

The present invention provides isolated Pseudo-ICE and ICE-Like, functional fragments thereof, or immunogenic fragments thereof and nucleic acid molecules encoding the above polypeptides. Also provided are various methods of using these polypeptides or nucleic acid molecules in modulating apoptosis or inflammation.

### STATEMENT REGARDING FEDERALLY SPONSORED RESEARCH OR DEVELOPMENT

This invention was made in part with funds provided by the United States Government under National Institutes of Health Research Grant AG14357. Accordingly, the United States Government may have certain rights to this invention.

### BACKGROUND OF THE INVENTION

1. Field of the Invention

This invention relates generally to polynucleic acids and products encoded thereby which regulate apoptosis, cell proliferation and inflammation. More specifically, this invention discloses two caspase recruitment domain (CARD) polypeptides and nucleic acids encoding the same. This invention also discloses methods of using these polypeptides and/or encoding nucleic acids thereof to mediate apoptosis, cell proliferation and/or inflammation and to identify effectors for various processes involved in apoptosis, cell proliferation and/or inflammation.

2. Description of the Related Art

Interleukin-1 (“IL-1”) is a major pro-inflammatory and immunoregulatory protein that stimulates fibroblast differentiation and proliferation, the production of prostaglandins, collagenase and phospholipase by synovial cells and chondrocytes, basophil and eosinophil degranulation and neutrophil activation (Oppenheim et al., Immunol. Today 7:45-56 (1986)). As such, it is involved in the pathogenesis of chronic and acute inflammatory and autoimmune diseases. IL-1 is predominantly produced by peripheral blood monocytes as part of the inflammatory response (Mosely, et al., Proc. Nat. Acad. Sci. 84:4572-4576 (1987); Lonnemann, et al., Eur. J. Immunol, 19:1531-1536 (1989)).

IL-1β is synthesized as a biologically inactive precursor, proIL-1β. ProIL-1β is cleaved by a cysteine protease called interleukin-1β converting enzyme (“ICE”, also referred to as “caspase-1”) between Asp-116 and Ala-117 to produce the biologically active C-terminal fragment found in human serum and synovial fluid (Sleath, et al., J. Biol. Chem., 256:14526-14528 (1992); A. D. Howard, et al., J. Immunol., 147:2964-2969 (1991)).

ICE is a cysteine protease localized primarily in monocytes. It is synthesized as a single-chain polypeptide zymogen (referred to as “pro-caspase-1” or “pro-ICE”) consisting of an N-terminal fragment, and a large (p20) and a small (p10) catalytic domain (Thornberry et al., Nature 356:768-74 (1992)). Pro-ICE has low but detectable enzymatic activity. Upon receipt of a proinflammatory signal, pro-ICE is thought to oligomerize and autoprocess to generate the active p10/p20 herterodimeric protease (Walker et al., Cell 78:343-52 (1994); Wilson et al., Nature 370:270-5 (1994)). In addition to promoting the pro-inflammatory and immunoregulatory properties of IL-1β, ICE and particularly ICE homologues, also appear to be involved in the regulation of cell death or apoptosis (Yuan, et al., Cell 75:641-652 (1993); Miura, et al., Cell 75:653-660 (1993); Nett-Giordalisi, et al., J. Cell Biochem. 17B: 117 (1993)).

Apoptosis, also referred to as physiological cell death or programmed cell death, is a normal physiological process of cell death that plays a critical role in the regulation of tissue homeostasis by ensuring that the rate of new cell accumulation produced by cell division is offset by a commensurate rate of cell loss due to death. The process of apoptosis maintains tissue homeostasis in various physiological processes, including embryonic development, immune cell regulation, and normal cell turnover. It follows that loss of apoptosis can lead to a variety of pathological disease states. For example, the inappropriate loss of apoptosis can lead to the pathological accumulation of self-reactive lymphocytes such as those occurring in association with many autoimmune diseases. Inappropriate loss of apoptosis can also lead to the accumulation of virally infected cells and of hyperproliferative cells such as tumor cells. Similarly, the inappropriate activation of apoptosis can contribute to a variety of pathological disease states including, for example, acquired immunodeficiency syndrome (AIDS), neurodegenerative diseases and ischemic injury. Treatments that are specifically designed to modulate the apoptotic pathways in these and other pathological conditions can change the natural progression of many of these diseases.

Although apoptosis is mediated by diverse signals and complex interactions of cellular gene products, the results of these interactions ultimately feed into a cell death pathway that is evolutionarily conserved between humans and invertebrates (reviewed in Ashkenazi and Dixit, Science 281:1305-1308 (1998)). The pathway, itself, is a cascade of proteolytic events analogous to that of the blood coagulation cascade.

Several gene families and products that modulate the apoptotic process have been identified. One family is the NF-κB/Rel transcription factor family that includes, for example, RelA/p65, c-rel, RelB, p50 and p52. Transcriptionally active NF-κB is a homo- or hetero-dimer of two subunits of the NF-κB/Rel family. NF-κB is a critical regulator of cellular response to infectious agents, stress, injury and inflammation (reviewed in Verma et al., Genes Dev., 9:2723-2735 (1995); Baeuerle and Baltimore, Cell 87:13-20 (1996); Magnani et al., Curr. Drug Targets 1:387-99 (2000)). NF-κB has also been implicated in the regulation of apoptosis. For example, NF-κB is activated by certain apoptotic stimuli (Baldwin, Annu. Rev. Immunol,, 14:649 (1996)), and NF-κB directly regulates the expression of specific apoptotic genes, such as cIAP-1, cIAP-2, TRAF1 and TRAF2 (May and Ghosh, Immunol. Today 19:80-88 (1998); Wang, et al., Science 281:1680-1683). In resting cells NF-κB resides in the cytoplasm in inactive form, complexed to members of a family of inhibitory proteins referred to as IκB. The bound IκB masks the NF-κB nuclear localization signal and thereby inhibits its nuclear transportation. NF-κB can be activated by a variety of signals including inflammatory cytokines, bacterial lipopolysaccharides (LPS), oxidative and fluid mechanical stress. Upon activation by these stimuli, IκB is phosphorylated and subsequently degraded, which leads to NF-κB nuclear translocation and activation. Activated NF-κB regulates the expression of many genes involved in the immune and inflammatory responses, oncogenesis, as well as apoptosis.

Members of the caspase family of cysteine proteases play a central role in cell apoptosis. The human caspase family includes, for example, Ced-3, human ICE (interleukin-1β converting enzyme, caspase-1), ICH-1 (caspase-2), CPP32 (caspase-3), ICEᵣₑₗII (caspase-4), ICEᵣₑₗIII (caspase-5), Mch2 (caspase-6), ICE-LAP3 (caspase-7), Mch5 (caspase-8), ICE-LAP6 (caspase-9), Mch4 (caspase-10), caspase 11, 12, 13, 14 and others. The caspases are produced as inactive zymogens that require proteolytic processing to yield two subunits that associate to form an active heterodimer. The caspase targets include caspases themselves and a number of other cellular proteins. The caspases are activated sequentially in response to proapoptotic stimuli, resulting in the proteolytic cleavage of cellular targets and the characteristic morphological changes and DNA fragmentation associated with apoptosis (see Thornberry and Lazebnik, Science 281:1312-1316 (1998); Salveson and Dixit, Cell 91:443-446 (1997)).

Signals are relayed from the non-enzymatic upstream components of the apoptotic machinery to the downstream effector components via homotypic and heterotypic interactions between functional domains present in the molecules. For example, the caspase recruitment domain (CARD), which forms six antiparallel helices, mediates interactions between initiator caspases and death-inducing signaling complexes (Hofman, et al., Trends Biochem. Sci. 257:155-156 (1997); Chou, et al., Cell 94:171-180 (1998); Eberstadt et al., Nature 392:941-945 (1998)). In addition, the CARD is also present in the fragments at N termini of several caspases, such as caspase-1,-8 and -9 and involved in clustering mediated autoprocessing of these caspases (Srinivasula et al., Mol. Cell 1:1 (1998); Salvesen et al., Proc. Natl. Acad. Sci. USA 96:10964 (1999); Muzio et al., J. Biol. Chem. 273:2926 (1998)). For instance, caspase-8 possesses a CARD that allows the binding to the CARD of the adaptor FADD which itself associates with the death receptor Fas (Muzio et al., J. Biol. Chem. 273:2926 (1998)). The induced-proximity of several caspase-8 zymogen forms facilitates the autoprocessing of this protease (Srinivasula et al., Mol. Cell 1:1 (1998)). Similarly, the multimeric Apaf-1/cytochrome c/dATP complex induces caspase-9 activation via direact interaction between their respective CARD domains (Salvesen et al., Proc. Natl. Acad. Sci. USA 96:10964 (1999)).

Apoptosis is a fundamental cellular process regulating tissue homeostasis that has been implicated in a variety of human disease. Thus, there exists a need to identify apoptotic genes and their gene products and for methods of diagnosing and treating diseases associated with apoptosis. Furthermore, CARD-domain containing proteins play important roles in the cellular signaling processes that drive apoptosis. Therefore, proteins containing CARD domains are molecular targets of therapeutic intervention for disease-associated apoptosis.

Additionally, since IL-1β secretion results from caspase-1 (ICE) activation, inhibitors of pro-caspase-1 activation (i.e., processing of pro-caspase-1 to produce a p20/p10 heterodimeric protease) may be useful as therapeutic agents. Disease states in which inhibition of ICE activation may be useful include: infectious diseases, such as meningitis and salpingitis; septic shock, respiratory diseases; inflammatory conditions, such as arthritis, cholecystitis, colitis, encephalitis, endocerolitis, viral infections (e.g., hemoragghic fever, hepatitis, pancreatitis, etc.) and reperfusion injury; ischemic diseases such as myocardial infarction, stroke and ischemic kidney disease; immune-based diseases, such as hypersensitivity; autoimmune disease, such as multiple sclerosis; bone diseases; and certain neurodegenerative diseases, such as Alzheimer's and Parkinson's disease. Such inhibitors are useful for the repopulation of hematopoietic cells following chemotherapy and radiation therapy and for prolonging organ viability for use in transplantation.

Accordingly, the need exists for compounds that can effectively inhibit the action of the ICE family of proteases, for use as agents for preventing unwanted apoptosis, and for treating chronic and acute forms of IL-1 mediated diseases, such as inflammatory, autoimmune or neurodegenerative diseases. The present invention satisfies this need and provides further related advantages, such as providing diagnostic methods for diseases or disorders associated with abnormal expression of genes encoding CARD proteins or abnormal functions of CARD proteins, as well as providing screening assays for compounds useful for preventing or treating the above diseases or disorders.

### SUMMARY OF THE INVENTION

In one aspect, the present invention provides an isolated nucleic acid molecule comprising a nuclei-, acid sequence encoding a Pseudo-ICE having at least 80% amino acid identity to SEQ ID NO: 1, an immunogenic fragment of the Pseudo-ICE, or a functional fragment of the Pseudo-ICE that has an activity selected from: specific binding to pro-caspase-1, specific binding to RICK, inhibiting induced IL-1β secretion, and stimulating NF-κB activation. In certain embodiments, the isolated nucleic acid molecule encodes the Pseudo-ICE of SEQ ID NO: 1. In some embodiments, the isolated nucleic acid molecule has a nucleotide sequence at least 98% identical to SEQ ID NO: 2. In a preferred embodiment, the isolated nucleic acid has a nucleotide sequence as set forth in SEQ ID NO: 2.

In another aspect, the present invention provides an isolated nucleic acid molecule comprising a nucleic acid sequence encoding an ICE-Like having at least 80% amino acid identity to SEQ ID NO: 3, an immunogenic fragment of the ICE-Like, or a functional fragment of the ICE-Like that has an activity of specific binding to pro-caspase-1 and/or inhibiting induced IL-1β secretion. In certain embodiments,

the isolated nucleic acid molecule encodes the ICE-Like of SEQ ID NO: 3. In some embodiments, the isolated nucleic acid molecule has a nucleotide sequence at least 98% identical to SEQ ID NO: 4. In a preferred embodiment, the isolated nucleic acid has a nucleotide sequence as set forth in SEQ ID NO: 4.

In another aspect, the present invention provides a nucleic acid vector comprising a nucleic acid sequence encoding a Pseudo-ICE having at least 80% amino acid identity to SEQ ID NO: 1 or a functional fragment of the Pseudo-ICE. In a preferred embodiment, the nucleic acid vector comprises a nucleic acid sequence encoding Pseudo-ICE that has an amino acid sequence set forth in SEQ ID NO: 1. In some embodiments, the nucleic acid vector comprises a nucleic acid sequence at least 98% identical to SEQ ID NO: 2. In certain preferred embodiments, the nucleic acid vector comprises a nucleotide sequence as set forth in SEQ ID NO: 2.

In another aspect, the present invention provides a nucleic acid vector comprising a nucleic acid sequence encoding an ICE-Like having at least 80% amino acid identity to SEQ ID NO: 1 or a functional fragment of the ICE-Like. In a preferred embodiment, the nucleic acid vector comprises a nucleic acid sequence encoding ICE-Like that has an amino acid sequence set forth in SEQ ID NO: 3. In some embodiments, the nucleic acid vector comprises a nucleic acid sequence at least 70%, 80%, 90%, 95% or 98% identical to SEQ ID NO: 4. In certain preferred embodiments, the nucleic acid vector comprises a nucleotide sequence as set forth in SEQ ID NO: 4.

In another aspect, the present invention provides a host cell contain the nucleic acid vector described above. The host cell may be selected from the group consisting of a mammalian ceil, a plant cell, an insect cell, a yeast cell and a bacterial cell.

In another aspect, the present invention provides an isolated polypeptide comprising a Pseudo-ICE having at least 80% amino acid identity to SEQ ID NO: 1, an immunogenic fragment of the Pseudo-ICE, or a functional fragment of the Pseudo-ICE that has an activity selected from: specific binding to pro-caspase-1, specific binding to RICK, inhibiting induced IL-1β secretion, and stimulating NF-κB activation. In a preferred embodiment, the isolated polypeptide comprises a Pseudo-ICE of SEQ ID NO: 1.

In a related aspect, the present invention provides an isolated polypeptide encoded by a nucleotide sequence at least 98% identical to SEQ ID NO: 2 and having an activity selected from: specific binding to pro-caspase-1, specific binding to RICK, inhibiting induced IL-1β secretion, and stimulating NF-κB activation.

In another aspect, the present invention provides an isolated polypeptide comprising an ICE-Like having at least 80% amino acid identity to SEQ ID NO: 3, an immunogenic fragment of the ICE-Like, or a functional fragment of the ICE-Like that is capable of specific binding to pro-caspase-1 and/or inhibiting induced IL-1β secretion. In a preferred embodiment, the isolated polypeptide comprises an ICE-Like of SEQ ID NO: 3.

In a related aspect, the present invention provides an isolated polypeptide encoded by a nucleotide sequence at least 98% identical to SEQ ID NO: 4 and having an capability of specific binding to pro-caspase-1 and/or inhibiting induced IL-1β secretion.

In another aspect, the present invention provides an antibody comprising an immnunoglobulin or antigen-binding fragment thereof that specifically binds to a Pseudo-ICE or to an immunogenic or functional fragment thereof.

In another aspect, the present invention provides an antibody comprising an immunoglobulin or antigen-binding fragment thereof that specifically binds to an ICE or to an immunogenic or functional fragment thereof.

In one aspect, the present invention provides a composition comprising the isolated Pseudo-ICE or an immunogenic or functional fragment thereof and a physiologically acceptable carrier.

In another aspect, the present invention provides a composition comprising the isolated ICE-Like or an immunogenic or functional fragment thereof and a physiologically acceptable carrier.

In another aspect, the present invention provides a method of transforming or transfecting a cell with a nucleic acid molecule encoding a Pseudo-ICE that has at least 80% amino acid identity to SEQ ID NO: 1 or a functional fragment of the Pseudo-ICE that has an activity selected from: specific binding to pro-caspase-1, specific binding to RICK, inhibiting induced IL-1β secretion, and stimulating NF-κB activation, comprising contacting the cell with a vector comprising the nucleic acid molecule under the control of a promoter. In certain embodiments, the Pseudo-ICE of the above method has an amino acid sequence set forth in SEQ ID NO: 1. In some embodiments, the nucleic acid molecule encoding a Pseudo-ICE is at least 98% identical to SEQ ID NO: 2. In a preferred embodiment, the nucleic acid molecule has a nucleotide sequence set forth in SEQ ID NO: 2. The nucleic acid molecule encoding a Pseudo-ICE may be in the sense or antisense orientation with respect to the promoter.

In another aspect, the present invention provides a method of transforming or transfecting a cell with a nucleic acid molecule encoding an ICE-Like that has at least 80% amino acid identity to SEQ ID NO: 1 or a functional fragment of the Pseudo-ICE that has an activity to specifically bind to pro-caspase-1 and/or to inhibit induced IL-1β secretion, comprising contacting the cell with a vector comprising the nucleic acid molecule under the control of a promoter. In certain embodiments, the ICE-Like of the above method has an amino acid sequence set forth in SEQ ID NO: 3. In some embodiments, the nucleic acid molecule encoding a ICE is at least 70%, 80%, 90%, 95%, or 98% identical to SEQ ID NO: 2. In a preferred embodiment, the nucleic acid molecule has a nucleotide sequence set forth in SEQ ID NO: 4. The nucleic acid molecule encoding an ICE-Like may be in the sense or antisense orientation with respect to the promoter.

In another aspect, the present invention provides a method of inhibiting apoptosis or inflammation comprising contacting a cell expressing a pro-caspase-1 with a composition comprising the isolated polypeptide described above (i.e., polypeptides comprising full length, an immunogenic fragment or a functional fragment of Pseudo-ICE or ICE-Like) under conditions and for a time sufficient to permit the inhibition of apoptosis.

In another aspect, the present invention provides a method of inhibiting apoptosis or inflammation comprising contacting a cell expressing a pro-caspase-1 with a composition comprising the isolated nucleic acid molecule described above (i.e., nucleic acid molecules comprising a nucleic acid sequence encoding full length, an immunogenic fragment or a functional fragment of Pseudo-ICE or ICE-Like) under conditions and for a time sufficient to permit the inhibition of apoptosis.

In another aspect, the present invention provides a method of stimulating apoptosis comprising contacting a cell expressing a pro-caspase-1 a composition comprising a polypeptide that specifically binds to the isolated polypeptide described above under conditions and for a time sufficient to permit the stimulation of apoptosis. In a preferred embodiment, the polypeptide that specifically binds to the isolated polypeptide described above is an immunoglobulin or antigen-binding fragment that specifically binds to the isolated polypeptide.

The present invention also provides a method of stimulating apoptosis comprising contacting a cell expressing a pro-caspase-1 a composition comprising an antisense or ribozyme construct of the isolated nucleic acid molecule described above under conditions and for a time sufficient to permit the stimulation of apoptosis.

The present invention also provides a method of inhibiting the activation of a pro-caspase-1 comprising contacting a cell expressing the pro-caspase-1 with a composition comprising the isolated polypeptide described above under conditions and for a time sufficient to permit the inhibition of the activation of the pro-caspase-1.

The present invention also provides a method of inhibiting the activation of a pro-caspase-1 comprising contacting a cell expressing the pro-caspase-1 with a composition comprising the isolated nucleic acid molecule described above under conditions and for a time sufficient to permit the inhibition of the activation of the pro-caspase-1.

The present invention also provides a method of stimulating the activation of a pro-caspase-1 comprising contacting a cell expressing the pro-caspase-1 a composition comprising a polypeptide that specifically binds to the isolated polypeptide described above under conditions and for a time sufficient to permit the stimulation of the activation of the pro-caspase-1. In a preferred embodiment, the polypeptide is an immunoglobulin or antigen-binding fragment thereof that specifically binds to the isolated polypeptide described above.

The present invention also provides a method of stimulating the activation of a pro-caspase-1 comprising contacting a cell expressing the pro-caspase-1 a composition comprising an antisense or ribozyme construct of the isolated nucleic acid molecule described above under conditions and for a time sufficient to permit the stimulation of the activation of the pro-caspase-1.

The present invention also provides a method of identifying inhibitors or enhancers of Pseudo-ICE mediated inhibition of pro-caspase-1 activation, comprising: contacting a cell transfected with an expression vector encoding Pseudo-ICE having at least 80% amino acid identity to SEQ ID NO: 1 or a functional fragment of the Pseudo-ICE capable of inhibiting pro-caspase-1 activation with a candidate inhibitor or enhancer; and detecting an increase or decrease in pro-caspase-1 activation in the presence of the candidate inhibitor or enhancer, wherein a decrease in pro-caspase-1 activation indicates the presence of an enhancer of Pseudo-ICE mediated inhibition of pro-caspase-1 activation and an increase in pro-caspase-1 activation indicates the presence of an enhancer.

The level of pro-caspase-1 activation may be determined by measuring the level of pro-caspase-1 oligomerization, the level of pro-caspase-1 processing activity, the substrate cleavage activity of the caspase-1 processed from the pro-caspase-1, and the level of IL-1β secretion induced by an inflammatory stimulus.

The present invention also provides a method of identifying inhibitors or enhancers of ICE-Like mediated inhibition of pro-caspase-1 activation, comprising: contacting a cell transfected with an expression vector encoding Pseudo-ICE having at least 80% amino acid identity to SEQ ID NO: 1 or a functional fragment of the Pseudo-ICE capable of inhibiting pro-caspase-1 activation with a candidate inhibitor or enhancer; and detecting an increase or decrease in pro-caspase-1 activation in the presence of the candidate inhibitor or enhancer, wherein a decrease in pro-caspase-1 activation indicates the presence of an enhancer of Pseudo-ICE mediated inhibition of pro-caspase-1 activation and an increase in pro-caspase-1 activation indicates the presence of an enhancer.

The level of pro-caspase-1 activation may be determined by measuring the level of pro-caspase-1 oligomerization, the level of pro-caspase-1 processing activity, the substrate cleavage activity of the caspase-1 processed from the pro-caspase-1, and the level of IL-1β secretion induced by an inflammatory stimulus.

The present invention also provides a method of inhibiting induced secretion of IL-1β comprising contacting a cell subject to an inflammatory stimulus that induces IL-1β secretion with a composition comprising the isolated polypeptide described above (i.e., polypeptides comprising a full length, an immunogenic fragment or a functional fragment of Pseudo-ICE or ICE-Like) under conditions and for a time sufficient to permit the inhibition of IL-1β secretion induced by the stimulus.

The present invention also provides a method of inhibiting induced secretion of IL-1β comprising contacting a cell subject to an inflammatory stimulus that induces IL-1β secretion with a composition comprising the isolated nucleic acid molecule described above (i.e., nucleic acid molecules comprising a nucleic acid sequence encoding a full length, an immunogenic fragment or a functional fragment of Pseudo-ICE or ICE-Like) under conditions and for a time sufficient to permit the inhibition of IL-1β secretion induced by the stimulus. In certain embodiments, the inflammatory stimulus is an IFN-γ, a TNF, or a lipopolysaccharide.

The present invention also provides a method of identifying inhibitors or enhancers of Pseudo-ICE mediated inhibition of IL-1β secretion in response to an inflammatory stimulus, comprising: contacting a cell transfected with an expression vector encoding Pseudo-ICE having at least 80% amino acid identity to SEQ ID NO: 1 or a functional fragment of the Pseudo-ICE capable of inhibiting the IL-1β secretion with a candidate inhibitor or enhancer; subjecting the transfected cell to an inflammatory stimulus; and detecting an increase or decrease in the IL-1β secretion level induced by the inflammatory stimulus in the presence of the candidate inhibitor or enhancer, wherein a decrease in the induced IL-1β secretion indicates the presence of an enhancer of Pseudo-ICE mediated inhibition of the induced IL-1β secretion and an increase in the induced IL-1β secretion indicates the presence of an inhibitor of Pseudo-ICE mediated inhibition of the induced IL-1β secretion.

In certain embodiments, the inflammatory stimulus is an IFN-γ, a TNF, or a lipopolysaccharide. In a preferred embodiment, the Pseudo-ICE has an amino acid sequence set forth in SEQ ID NO: 1.

The present invention also provides a method of identifying inhibitors or enhancers of ICE-mediated inhibition of IL-1β secretion in response to an inflammatory stimulus, comprising: contacting a cell transfected with an expression vector encoding Pseudo-ICE having at least 80% amino acid identity to SEQ ID NO: 3 or a functional fragment of the ICE-Like capable of inhibiting the IL-1β secretion with a candidate inhibitor or enhancer; subjecting the transfected cell to an inflammatory stimulus; and detecting an increase or decrease in the IL-1β secretion level induced by the inflammatory stimulus in the presence of the candidate inhibitor or enhancer, wherein a decrease in the induced IL-1β secretion indicates the presence of an enhancer of ICE-Like mediated inhibition of the induced IL-1β secretion and an increase in the induced IL-1β secretion indicates the presence of an inhibitor of ICE-Like mediated inhibition of the induced IL-1β secretion.

In certain embodiments, the inflammatory stimulus is an IFN-γ, a TNF, or a lipopolysaccharide. In a preferred embodiment, the Pseudo-ICE has an amino acid sequence set forth in SEQ ID NO: 1.

The present invention also provides a method of stimulating the activation of an NF-kB comprising contacting a cell expressing the NF-kB with a composition comprising an isolated polypeptide that comprises a full-length, an immunogenic fragment or a functional fragment of Pseudo-ICE under conditions and for a time sufficient to permit the inhibition of the activation of the NF-kB.

The present invention also provides a method of stimulating the activation of an NF-kB comprising contacting a cell expressing the NF-kB with a composition comprising an isolated nucleic acid molecule that comprises a nucleic acid sequence encoding a full-length, an immunogenic fragment or a functional fragment of Pseudo-ICE under conditions and for a time sufficient to permit the inhibition of the activation of the NF-kB.

The present invention also provides a method of inhibiting the activation of an NF-kB comprising contacting a cell expressing the NF-kB a composition comprising a polypeptide that specifically binds to the isolated polypeptide that comprises a full-length, an immunogenic fragment or a functional fragment of Pseudo-ICE under conditions and for a time sufficient to permit the stimulation of the activation of the NF-kB. In certain embodiment, the polypeptide is an immunoglobulin or antigen-binding fragment that specifically binds to Pseudo-ICE or a fragment thereof.

The present invention also provide a method of inhibiting the activation of an NF-kB comprising contacting a cell expressing the NF-kB a composition comprising an antisense or ribozyme construct of the isolated nucleic acid molecule that comprises a nucleic acid sequence encoding a full-length, an immunogenic fragment or a functional fragment of Pseudo-ICE under conditions and for a time sufficient to permit the stimulation of the activation of the NF-kB.

The present invention also provides a method of identifying inhibitors or enhancers of Pseudo-ICE mediated NF-kB activation, comprising: contacting a cell transfected with an expression vector encoding Pseudo-ICE having at least 80% amino acid identity to SEQ ID NO: 1 or a functional fragment of the Pseudo-ICE capable of stimulating NF-κB activation with a candidate inhibitor or enhancer; and detecting an increase or decrease in NF-kB activation in the presence of the candidate inhibitor or enhancer, wherein a decrease in NF-kB activation indicates the presence of an inhibitor and an increase in NF-kB indicates the presence of an enhancer.

In a preferred embodiment, the Pseudo-ICE has an amino acid sequence set forth in SEQ ID NO: 1.

The present invention also provides a method of identifying a polypeptide that specifically binds to a Pseudo-ICE having at least 80% amino acid identity to SEQ ID NO: 1 or a functional fragment of the Pseudo-ICE that has an activity selected from: specific binding to pro-caspase-1, specific binding to RICK, inhibiting induced IL-1β secretion, and stimulating NF-κB activation, comprising: contacting a sample with the Pseudo-ICE or the functional fragment under conditions that permit the formation of a complex between the Pseudo-ICE or the functional fragment thereof and the polypeptide; and detecting the complex and polypeptide in the complex.

In a preferred embodiment, the Pseudo-ICE has an amino acid sequence set forth in SEQ ID NO: 1. In certain embodiments, the Pseudo-ICE or the functional fragment thereof is covalently bound to a detectable moiety, such as a reporter molecule, a radionuclide. In some embodiments, the sample comprises a cDNA expression library.

The present invention also provides a nucleic acid molecule comprising a first nucleic acid sequence encoding a Pseudo-ICE having at least 80% identity to SEQ ID NO: 1 or a functional fragment of the Pseudo-ICE that has an activity selected from: specific binding to pro-caspase-1, specific binding to RICK, inhibiting induced IL-1β secretion, and stimulating NF-κB activation and a second nucleic acid sequence encoding the transcription activation domain or the DNA-binding domain of a transcription factor.

In a related aspect, the present invention also provides a method for identifying a polypeptide that specifically binds to a Pseudo-ICE having at least 80% amino acid identity to SEQ ID NO: 1 or a functional fragment of the Pseudo-ICE that has an activity selected from: specific binding to pro-caspase-1, specific binding to RICK, inhibiting induced IL-1β secretion, and stimulating NF-κB activation with a yeast two-hybrid screening system, comprising transforming a yeast cell with a vector comprising the nucleic acid molecule described above.

The present invention also provides a nucleic acid molecule comprising a first nucleic acid sequence encoding an ICE-Like having at least 80% identity to SEQ ID NO: 3 or a functional fragment of the ICE-Like that has an activity of specific binding to pro-caspase-1 and/or inhibiting induced IL-1β secretion and a second nucleic acid sequence encoding the transcription activation domain or the DNA-binding domain of a transcription factor.

In a related aspect, the present invention also provides a method for identifying a polypeptide that specifically binds to an ICE-Like having at least 80% amino acid identity to SEQ ID NO: 3 or a functional fragment of the ICE-Like that has an activity of specific binding to pro-caspase-1 and/or inhibiting induced IL-1β secretion, with a yeast two-hybrid screening system, comprising transforming a yeast cell with a vector comprising the nucleic acid molecule described above.

The present invention also provides a method for identifying a compound that increases the specific binding between a pro-caspase-1 or a fragment thereof that comprises a pro-domain of the pro-caspase-1 and a Pseudo-ICE having at least 80% amino acid identity to SEQ ID NO: 1 or a functional fragment of Pseudo-ICE capable of binding to the pro-caspase-1 or the fragment thereof, comprising: combining the Pseudo-ICE or the functional fragment with the pro-caspase-1 or the fragment thereof in the absence of a candidate compound under conditions that allow specific binding between the Pseudo-ICE or the functional fragment and the pro-capase-1 or the fragment thereof; combining the Pseudo-ICE or the functional fragment with the pro-caspase-1 or the fragment thereof in the presence of the candidate compound under the same conditions; comparing the specific binding between the Pseudo-ICE or the functional fragment and the pro-caspase-1 or the fragment thereof in the absence of the candidate compound with that in the presence of the candidate compound to thereby determine whether the candidate compound is capable of increasing the specific binding between the Pseudo-ICE or the functional fragment and the pro-caspase-1 or the fragment thereof.

In certain embodiments, the Pseudo-ICE has an amino acid sequence of SEQ ID NO: 1.

In a related aspect, the present invention also provides a process for manufacturing a compound that increases the specific binding between a pro-caspase-1 or a fragment thereof that comprises a pro-domain of the pro-caspase-1 and a Pseudo-ICE having at least 80% amino acid identity to SEQ ID NO: 1 or a functional fragment of Pseudo-ICE capable of binding to the pro-caspase-1 or the fragment thereof, comprising: carrying out the above method to identify a compound that increases the specific binding between a pro-caspase-1 or the fragment thereof and a Pseudo-ICE having at least 80% amino acid identity to SEQ ID NO: 1 or a functional fragment of Pseudo-ICE capable of binding to the pro-caspase-1; derivatizing the compound; and optionally repeating the above two steps.

In certain embodiments, the Pseudo-ICE has an amino acid sequence of SEQ ID NO: 1.

The present invention also provides a method for identifying a compound that increases the specific binding between a pro-caspase-1 or a fragment thereof that comprises a pro-domain of the pro-caspase-1 and an ICE-Like having at least 80% amino acid identity to SEQ ID NO: 3 or a functional fragment of Pseudo-ICE capable of binding to the pro-caspase-1 or the fragment thereof, comprising: combining the ICE-Like or the functional fragment with the pro-caspase-1 or the fragment thereof in the absence of a candidate compound under conditions that allow specific binding between the ICE-Like or the functional fragment and the pro-capase-1 or the fragment thereof, combining the ICE-Like or the functional fragment with the pro-caspase-1 or the fragment thereof in the presence of the candidate compound under the same conditions; and comparing the specific binding between the ICE-Like or the functional fragment and the pro-caspase-1 or the fragment thereof of in the absence of the candidate compound with that in the presence of the candidate compound to thereby determine whether the candidate compound is capable of increasing the specific binding between the ICE-Like or the functional fragment and the pro-caspase-1 or the fragment thereof.

In certain embodiments, the ICE-Like has an amino acid sequence of SEQ ID NO: 3.

In a related aspect, the present invention also provides a process for manufacturing a compound that increases the specific binding between a pro-caspase-1 or a fragment thereof that comprises a pro-domain of the pro-caspase-1 and an ICE-Like having at least 80% amino acid identity to SEQ ID NO: 3 or a functional fragment of ICE-Like capable of binding to the pro-caspase-1 or the fragment thereof, comprising: carrying out the above to identify a compound that increases the specific binding between a pro-caspase-1 or the fragment thereof and a Pseudo-ICE having at least 80% amino acid identity to SEQ ID NO: 3 or a functional fragment of ICE-Like capable of binding to the pro-caspase-1; derivatizing the compound; and optionally repeating the above two steps.

In certain embodiments, the ICE-Like has an amino acid sequence of SEQ ID NO: 3.

The present invention also provides a method for identifying a compound that decreases the specific binding between a pro-caspase-1 or a fragment thereof that comprises a pro-domain of the caspase-1 and a Pseudo-ICE having at least 80% amino acid identity to SEQ ID NO: 1 or a functional fragment of Pseudo-ICE capable of binding to a pro-caspase-1 or the fragment thereof, comprising: combining the Pseudo-ICE or the functional fragment with the pro-caspase-1 or the fragment thereof in the absence of a candidate compound under conditions that allow specific binding between the Pseudo-ICE or the functional fragment and the pro-capase-1 or the fragment thereof, combining the Pseudo-ICE or the functional fragment with the pro-caspase-1 or the fragment thereof in the presence of the candidate compound under the conditions and comparing the specific binding between the Pseudo-ICE or the functional fragment and the pro-caspase-1 or the fragment thereof in the absence of the candidate compound with that in the presence of the candidate compound to thereby determine whether the candidate compound is capable of decreasing the specific binding between the Pseudo-ICE or the functional fragment and the pro-caspase-1.

In certain embodiments, Pseudo-ICE has an amino acid sequence of SEQ ID NO: 1.

The present invention also provides a method for identifying a compound that decreases the specific binding between a pro-caspase-1 or a fragment thereof that comprises a pro-domain of the caspase-1 and an ICE-Like having at least 80% amino acid identity to SEQ ID NO: 3 or a functional fragment of ICE-Like capable of binding to a pro-caspase-1 or the fragment thereof, comprising: combining the ICE-Like or the functional fragment with the pro-caspase-1 or the fragment thereof in the absence of a candidate compound under conditions that allow specific binding between the ICE-Like or the functional fragment and the pro-capase-1 or the fragment thereof; combining the ICE-Like or the functional fragment with the pro-caspase-1 or the fragment thereof in the presence of the candidate compound under the same conditions; and comparing the specific binding between the ICE-Like or the functional fragment and the pro-caspase-1 or the fragment thereof in the absence of the candidate compound with that in the presence of the candidate compound to thereby determine whether the candidate compound is capable of decreasing the specific binding between the ICE-Like or the functional fragment and the pro-caspase-1.

In certain embodiments, ICE-Like has an amino acid sequence of SEQ ID NO: 3.

The present invention also provides a process for manufacturing a compound that decreases the specific binding between a pro-caspase-1 or a fragment thereof that comprises a pro-domain of the pro-caspase-1 and a Pseudo-ICE having at least 80% amino acid identity to SEQ ID NO: 1 or a functional fragment of Pseudo-ICE capable of binding to a pro-caspase-1 or the fragment thereof, comprising: carrying out the method described above to identify a compound that decreases the specific binding between the pro-caspase-1 or the fragment thereof and the Pseudo-ICE or the functional fragment of Pseudo-ICE; derivatizing the compound; and optionally repeating the above two steps.

In certain embodiments, the Pseudo-ICE has an amino acid sequence of SEQ ID NO: 1.

The present invention also provides a process for manufacturing a compound that decreases the specific binding between a pro-caspase-1 or a fragment thereof that comprises a pro-domain of the pro-caspase-1 and an ICE-Like having at least 80% amino acid identity to SEQ ID NO: 3 or a functional fragment of ICE-Like capable of binding to a pro-caspase-1 or the fragment thereof, comprising: carrying out the method described above to identify a compound that decreases the specific binding between the pro-caspase-1 or the fragment thereof and the ICE-Like or the functional fragment of ICE-Like; derivatizing the compound; and optionally repeating the above two steps.

In certain embodiments, the ICE-Like has an amino acid sequence of SEQ ID NO: 3.

The present invention also provides a method for identifying a compound that disrupts the binding between a pro-caspase-1 or a fragment thereof that comprises a pro-domain of the pro-caspase-1 and a Pseudo-ICE having at least 80% amino acid identity to SEQ ID NO: 1 or a functional fragment of the Pseudo-ICE capable of binding to the pro-caspase-1 or the fragment thereof, comprising: contacting a candidate compound with a binding complex comprising the Pseudo-ICE or the functional fragment and the pro-caspase-1; and detecting the Pseudo-ICE, the functional fragment, the pro-caspase-1 or the fragment thereof that dissociates from the binding complex to thereby determine whether the candidate compound is capable of disrupting the binding between the pro-caspase-1 or the fragment thereof and the Pseudo-ICE or the functional fragment.

The present invention also provides a method for identifying a compound that disrupts the binding between a pro-caspase-1 or a fragment thereof that comprises a pro-domain of the pro-caspase-1 and an ICE-Like having at least 80% amino acid identity to SEQ ID NO: 3 or a functional fragment of the ICE-Like capable of binding to the pro-caspase-1 or the fragment thereof, comprising: contacting a candidate compound with a binding complex comprising the ICE-Like or the functional fragment and the pro-caspase-1; and detecting the ICE-Like, the functional fragment, the pro-caspase-1 or the fragment thereof that dissociates from the binding complex to thereby determine whether the candidate compound is capable of disrupting the binding between the pro-caspase-1 or the fragment thereof and the ICE-Like or the functional fragment.

In certain embodiments, the Pseudo-ICE or the functional fragment thereof, the ICE-Like or the functional fragment thereof, or the pro-caspase-1 or the fragment thereof is covalently bound to a detectable moiety, such as a reporter molecule or a radionuclide.

The present invention also provides a process for manufacturing a compound that disrupts the specific binding between a pro-caspase-1 or a fragment thereof that comprises and a Pseudo-ICE having at least 80% amino acid identity to SEQ ID NO: 1 or a functional fragment of Pseudo-ICE capable of binding to a pro-caspase-1 or the fragment thereof, comprising: carrying out the method described above to identify a compound that disrupts the specific binding between the pro-caspase-1 or the fragment thereof and the Pseudo-ICE or the functional fragment of Pseudo-ICE; derivatizing the compound; and optionally repeating the above two steps.

The present invention also provides a process for manufacturing a compound that disrupts the specific binding between a pro-caspase-1 or a fragment thereof that comprises a pro-domain of the pro-caspase-1 and an ICE-Like having at least 80% amino acid identity to SEQ ID NO: 3 or a functional fragment of ICE-Like capable of binding to a pro-caspase-1 or the fragment thereof, comprising: carrying out the method described above to identify a compound that disrupts the specific binding between the pro-caspase-1 or the fragment thereof and the ICE-Like or the functional fragment of ICE-Like; derivatizing the compound; and optionally repeating the above two steps.

The present invention also provides a method for identifying a compound that increases the specific binding between a RICK and a Pseudo-ICE having at least 80% amino acid identity to SEQ ID NO: 1 or a functional fragment of Pseudo-ICE capable of binding to the RICK, comprising: combining the Pseudo-ICE or the functional fragment with the RICK in the absence of a candidate compound under conditions that allow specific binding between the Pseudo-ICE or the functional fragment and the capase-1; combining the Pseudo-ICE or the functional fragment with the RICK in the presence of the candidate compound under the same conditions; and comparing the specific binding between the Pseudo-ICE or the functional fragment and the RICK in the absence of the candidate compound with that in the presence of the candidate compound to thereby determine whether the candidate compound is capable of increasing the specific binding between the Pseudo-ICE or the functional fragment and the RICK.

The present application also provides a method for identifying a compound that decreases the specific binding between a RICK and a Pseudo-ICE having at least 80% amino acid identity to SEQ ID NO: 1 or a functional fragment of Pseudo-ICE capable of binding to a RICK, comprising: combining the Pseudo-ICE or the functional fragment with the RICK in the absence of a candidate compound under conditions that allow specific binding between the Pseudo-ICE or the functional fragment and the RICK; combining the Pseudo-ICE or the functional fragment with the RICK in the presence of the candidate compound under the same conditions; and comparing the specific binding between the Pseudo-ICE or the functional fragment and the RICK in the absence of the candidate compound with that in the presence of the candidate compound to thereby determine whether the candidate compound is capable of decreasing the specific binding between the Pseudo-ICE or the functional fragment and the RICK.

The present invention also provides a method for identifying a compound that disrupts the binding between a RICK and a Pseudo-ICE having at least 80% amino acid identity to SEQ ID NO: 1 or a functional fragment of the Pseudo-ICE capable of binding to the RICK, comprising: contacting a candidate compound with a binding complex comprising the Pseudo-ICE or the functional fragment and the RICK; and detecting the Pseudo-ICE, the functional fragment, or the RICK that dissociates from the binding complex to thereby determine whether the candidate compound is capable of disrupting the binding between the RICK and the Pseudo-ICE or the functional fragment.

In certain embodiments, the Pseudo-ICE has an amino acid sequence set forth in SEQ ID NO: 1. In some embodiments, the Pseudo-ICE, the functional fragment or the RICK is covalently bound to a detectable moiety such as a reporter molecule and a radionuclide.

These and other aspects of the present invention will become evident upon reference to the following detailed description and attached drawings. In addition, the various references set forth herein describe in more detail certain procedures or compositions (e.g., plasmids, etc.), and are therefore incorporated by reference in their entirety.

### BRIEF DESCRIPTION OF THE SEVERAL VIEWS OF THE DRAWINGS

FIGS. 1A, 1B, 1C, and 1D show multiple amino acid sequence alignment of a pro-domain of human pro-caspase-1/ICE-α (SEQ ID NO: 5), a Pseudo-ICE (SEQ ID NO: 1), and an ICE-Like (SEQ ID NO: 3) (Panel A); colinear alignment of nucleotide sequences of the pro-domain of pro-caspase-1/ICE-α (SEQ ID NO: 6) and the Pseudo-ICE (SEQ ID NO: 7), including the 3′ untranslated region of the Pseudo-ICE (Panel B); the putative structure of the Pseudo-ICE gene (SEQ ID NO: 8) (Panel C); and the distribution of Pseudo-ICE, ICE-Like and caspase-1 mRNA in tissues (upper panels of Panel D) and cell lines (lower panels of Panel D).

FIGS. 2A, 2B and 2C show in vitro and in vivo interactions of Pseudo-ICE and ICE-Like with pro-caspase-1. Panel A shows SDS-polyacrylamide gel analysis of purified GST, Pseudo-ICE-GST and ICE-Like-GST proteins. Panel B is autoradiography of SDS-polyacrylamide gel analysis of in vitro protein-protein interaction between in vitro translated ³⁵S-labeled Pseudo-ICE, ICE-Like or pro-caspase-1 C to A (Casp-1 C/A) and Pseudo-ICE-GST, GST or ICE-Like-GST bound to gluthatione. Panel C shows western blot analysis of immunoprecipitates of 293 T cells transiently cotransfected with expression constructs encoding T7-tagged Pseudo-ICE, ICE-Like, pro-caspase-1 C to A, or CRADD and Flag-tagged Pseudo-ICE or ICE-Like or an empty vector control precipitated with a monoclonal anti-Flag Ab and western blot analyses of the corresponding cellular lysates. The upper panel shows western blot analysis of the immunoprecipitates blotted with an HRP-conjugated anti-T7 Ab. The middle panel shows western blot analysis of the corresponding cellular lysates blotted with an HRP-conjugated anti-T7 Ab. The lower panel shows western blot analysis of the same cellular lysates blotted with an anti-Flag Ab followed by HRP-conjugated secondary Ab and chemiluminescence detection.

FIGS. 3A and 3B show in vivo interactions of Pseudo-ICE and ICE-Like with RICK. Panel A shows western blot analysis of proteins from 293 T cells cotransfected with equal amounts of pcDNA3 encoding T7-tagged Pseudo-ICE, ICE-Like, or pro-caspase-1 C to A (Casp-1 C/A) and Flag-tagged RICK immunoprecipitated with a monoclonal anti-Flag Ab and western blot analyses of the corresponding cellular lysates. The upper panel shows western blot analysis of the immunoprecipitates blotted with an HRP-conjugated anti-T7 Ab. The middle panel shows western blot analysis of the corresponding cellular lysates blotted with an HRP-conjugated anti-T7 Ab. The lower panel shows western blot analysis of the same cellular lysates blotted with an anti-Flag Ab followed by HRP-conjugated secondary Ab and chemiluminescence detection. Panel B shows western blot analysis of proteins from 293 T cells cotransfected with constructs encoding Flag-RICK and T7-caspase 1 C to A together with increasing amounts of T7-Pseudo-ICE or T7-ICE-Like constructs immunoprecipitated with a monoclonal anti-Flag Ab and western blot analyses of the corresponding cellular lysates. The upper panel shows western blot analysis of the immunoprecipitates blotted with an HRP-conjugated anti-T7 Ab. The middle panel shows western blot analysis of the corresponding cellular lysates blotted with an anti-Flag Ab followed by HRP-conjugated secondary Ab and chemiluminescence detection. The lower panel shows western blot analysis of the same cellular lysates blotted with an HRP-conjugated anti-T7 Ab.

FIGS. 4A and 4B show inhibition of IL-1-β secretion by Pseudo-ICE and ICE-Like. Panel A shows western blot analysis of T7-epitope tagged protein expression in THP-1 cells infected using a retroviral vector encoding T7-epitope tagged Pseudo-ICE, ICE-Like or pro-caspase-1 C to A (Casp-1 C/A). Panel B shows secreted IL-1β levels of THP-1 cells treated with or without IFN-γ and LPS.

FIG. 5 shows effect of Pseudo-ICE and ICE-Like on spontaneous and TNF-α-induced apoptosis. The upper panel shows percentages of apoptotic cells in MCF-7 Fas cells transiently transfected with pcDNA3 vector encoding T7-epitope tagged Pseudo-ICE, ICE-Like or caspase-9 (Casp-9) C to A together with pRSC-lacZ. The lower panel shows western blot analysis of the lysates of the above cells bloted with an HRP-conjugated anti-T7 Ab.

FIGS. 6A and 6B show effect of ectopic expression of Pseudo-ICE and ICE-Like on NF-κB activation. Panel A shows fold induction of NF-κB activity of 293 T cells transiently cotransfected with expression constructs encoding T7- or Flag-epitope tagged proteins together with NF-κB luciferase reporter construct and pRSC-lacZ treated with TNF-α compared to that of 293 T cells transfected with empty vector and without TNF-α treatment (the upper panel) and western blot analysis of the corresponding cell lysates using an HRP-conjugated anti-T7 Ab (the lower panel). Panel B shows relative luciferase activities normalized for β-galactosidase activity of 293 T cells transfected with equal amount of pcDNA3 T7-Pseudo-ICE and with increasing quantities of pCMV2 Flag-kinase inactive mutant of IKK-α (mIKK-α) (the upper panel) and western blot analysis of the corresponding cell lysates using an HRP-conjugated anti-T7 Ab (the lower panel).

### DETAILED DESCRIPTION OF THE INVENTION

Prior to setting forth the invention, it may be helpful to establish definitions of certain terms that are used herein. Unless defined otherwise, all scientific and technical terms used have the same meaning as is commonly understood by one skilled in the art to which this invention belongs.

An “isolated nucleic acid molecule” refers to a polynucleotide molecule in the form of a separate fragment or as a component of a larger nucleic acid construct, which has been separated from its source cell (including the chromosome it normally resides in) at least once, and preferably in a substantially pure form. Nucleic acid molecules may be comprised of a wide variety of nucleotides, including DNA, RNA, nucleotide analogues, or combination thereof.

Within the context of the present invention, antibodies are understood to include monoclonal antibodies, polyclonal antibodies, anti-idiotypic antibodies, antibody fragments (e.g., Fab, and F(ab′)₂, Fᵥ variable regions, or complementarity determining regions) and humanized or primatized™ antibodies.

References to “Pseudo-ICE” herein are intended to include polypeptides of any origin that are substantially homologous to and that are biologically equivalent to the Pseudo-ICE polypeptide characterized and described herein. More specifically, a Pseudo-ICE polypeptide of the present invention generally has an amino acid sequence at least 80%, 85%, 90%, 95%, 96%, 97%, 98%, 99% (including the percentages of all integer values between 80-99). However, Pseudo-ICE does not include pro-domains of pro-caspase-1 known to date. In addition to sequence identity to SEQ ID NO: 1, the Pseudo-ICE of the present invention is also able to interact with pro-caspase-1 or RICK, to inhibit IL-1β secretion induced by an inflammatory stimulus, or to induce NF-κB activation. In a preferred embodiment, the Pseudo-ICE of the present invention is able to interact with pro-caspase-1 and RICK, to inhibit IL-1β secretion induced by an inflammatory stimulus, and to induce NF-κB activation.

Within the context of this invention, a “Pseudo-ICE” includes a wild type Pseudo-ICE as well as a variant (including an allele) of the wild type polypeptide. Such a variant may result from natural polymorphisms or may be synthesized by recombinant methodology and differ from the wild type protein by one or more amino acid substitutions, insertions, deletions, or the like. Typically, when engineered, amino acid substitution will be conservative, i.e., substitutions of amino acids within groups of polar, non-polar, aromatic, or charged amino acids. The variant must retain at least one of functions of a wild-type Pseudo-ICE as described above. In a preferred embodiment, the variant retain all the functions of a wild-type Pseudo-ICE.

References to “ICE-Like” herein are intended to include polypeptides of any origin that are substantially homologous to and that are biologically equivalent to the ICE-Like polypeptide characterized and described herein. More specifically, an ICE-Like polypeptide of the present invention generally has an amino acid sequence at least 80%, 85%, 90%, 95%, 96%, 97%, 98%, 99% (including the percentages of all integer values between 80 and 99) identical to a human ICE-Like (SEQ ID NO: 3). In addition, ICE-Like is able to interact with pro-caspase-1 or to inhibit IL-1β secretion induced by an inflammatory stimulus. However, unlike Pseudo-ICE, ICE-Like is unable to interact with RICK or induce NF-κB activation. Similar to Pseudo-ICE, ICE-Like polypeptides also include variants of a wild type ICE-Like that are able to interact with pro-caspase-1 or inhibit IL-1β secretion. In a preferred embodiment, the variants are able to interact with pro-caspase-1 and inhibit IL-1β secretion.

As used herein, percent identity of two amino acid sequences or of two nucleic acids is determined using BLAST programs of Altschul et al. (J. Mol. Biol. 215: 403-10, 1990) with their default parameters. These programs implement the algorithm of Karlin and Altschul (Proc. Natl. Acad. Sci. USA 87:2264-8, 1990) modified as in Karlin and Altschul (Proc. Natl. Acad. Sci. USA 90:5873-7, 1993). BLAST programs are available, for example, at the web site http://www.ncbi.nlm.nih.gov.

A “functional fragment,” as used herein, refers to a fragment of the full-length Pseudo-ICE or ICE-Like polypeptide that retains at least one functional activity associated with a full-length Pseudo-ICE or ICE-Like, respectively. For instance, a functional fragment of Pseudo-ICE must retain at least one of the following functions: the ability to interact with pro-caspase-1, to interact with RICK, to inhibit IL-1β inhibition induced by an inflammatory stimulus, and to induce NF-κB activation. Likewise, a functional fragment of ICE-Like polypeptide must retain at least one of the following functions: the ability to interact with pro-caspase-1 and to inhibit IL-1β secretion induced by an inflammatory stimulus.

A molecule is said to “specifically bind” to a particular polypeptide (e.g., pro-caspase-1, Pseudo-ICE, ICE-like, or RICK) if it binds at a detectable level with the particular polypeptide, but does not bind detectably with another polypeptide containing an unrelated sequence. An “unrelated sequence,” as used herein, refers to a sequence that is at most 10% identical to a reference sequence (e.g., a pro-domain of pro-caspase-1, Pseudo-ICE, ICE-like, or RICK).

A “caspase” refers to a cysteine protease with specificity for substrate cleavage at Asp-X bonds, where “X” is an amino acid.

Caspase-1 is a specific member of the caspase family that is capable of converting an inactive precursor IL-1β to a mature proinflammatory cytokine. It is synthesized as a single-chain polypeptide zymogen (referred to as “pro-caspase-1” or “pro-ICE”) consisting of an N-terminal fragment, a large domain and a small domain. Pro-caspase-1 can be activated or processed in response to an inflammatory signal to generate a heterodimer of the large domain and the small domain that has protease activities. As used herein, the heterodimer having catalytic activities is referred to as “caspase-1.” Within the context of this invention, a “caspase-1” (or “pro-caspase-1”) includes a naturally occurring caspase-1 (or pro-caspase-1) as well as a variant of the naturally occurring caspase-1 (or pro-caspase-1) that consists essentially of the sequence of the naturally occurring caspase-1 (or pro-caspase-1). Exemplary naturally occurring pro-caspase-1 includes human caspase-1α (GenBank Acc. No. XP_{—}040782), human pro-caspase-1β (GenBank Acc. No. XP_{—}055386), and human pro-capase-1γ (GenBank Acc. No. XP_{—}055388). Both the naturally occurring caspase-1 and a variant thereof must have the activity of converting an inactive precursor IL-1β to a mature proinfiammatory cytokine.

A “pro-domain” of a caspase-1, as used herein, refers to a fragment of caspase-1 that is involved in pro-caspase-1 oligomerization. It does not contain the sequences within the large or small domain of the caspase-1. A pro-domain of pro-caspase-1 may contain all the sequence of the pro-caspase-1 other than those of the large and small domains. Alternatively, it may contain a portion of the sequence of pro-caspase-1 outside the large and small domains that is sufficient for the specific binding between that portion and the full-length pro-caspase-1.

A “RICK”, also referred to as “RIP2” or “CARDIAK,” is a polypeptide that is capable of binding to and activating caspase-1. It contains a serine/threonine kinase domain and a carboxy-terminal caspase-recruiting domain. An exemplary RICK includes human RICK (GenBank Acc. No. XP_{—}005312). Within the context of this invention, a “RICK” includes a naturally occurring RICK as well as a variant of the naturally occurring RICK that consists essentially of the sequence of the naturally occurring RICK. Both the naturally occurring RICK and a variant thereof must have the ability of specific binding to, and activating, pro-caspase-1.

A “NF-κB” is a transcription factor composed of dimeric complexes that binds the sequence 5′-GGGNYCCC-3′. It usually associated with members of the Rel family (e.g., p65, c-Rel, and Rel-B), which have potent transactivation domains. Within the context of this invention, a “NF-κB” includes a naturally occurring NF-κB and a variant of the naturally occurring NF-κB that consists essentially of the sequence of the naturally occurring NF-κB. The term “consist essentially of′ a NF-κB sequence, as used herein, refers to a sequence that is highly homologous to a NF-κB sequence so that the essential functions of the NF-κB have been retained in the sequence consisting essentially of the NF-κB.

As used herein, “carrier molecules” include polypeptides, nucleic acids, carbohydrates, lipids, natural or synthetic polymers, and other biologically or chemically active molecules that can produce compounds that may be administered to animals or humans without toxic effects.

A “nucleic acid vector” refers to a nucleic acid molecule such as a plasmid, cosmid, or bacteriophage that has the capability of replicating autonomously in a host cell, including a cloning vector and an expression vector. A cloning vector typically contains one or a small number of restriction endonuclease recognition sites at which foreign DNA sequences can be inserted in a determinable fashion without loss of essential biological function of the vector, as well as a marker gene suitable for use in identification and selection of cells transformed with the vector. An expression vector is a DNA molecule giving a gene that is expressed in a host cell. Typically gene expression is placed under the control of certain regulatory elements including promoters, tissue-specific regulatory elements and enhancers.

As used herein, “therapeutically effective amount” of a compound used for treating a particular disease is an amount sufficient to lessen any permanent or temporary symptoms associated with the disease. This amount may be administered in a single dose, or according to an effective regimen. The amount may extinguish all symptoms of the disease, however typically the disease symptoms will at least be reduced or ameliorated.

As used herein, “modulate” refers to the ability to alter from a basal level. Such alteration may increase or decrease the activity, and such activity may be a single specific event or multiple steps in a cascade pathway. As used in the context of apoptosis (e.g., “modulate apoptosis”), “modulate” refers to the ability to alter or change any biochemical, physiological or morphological events associated with apoptosis from its basal level. For example, apoptosis has been modulated if the expression of a gene involved in apoptotic pathway, the interaction of an apoptotic pathway protein with other proteins, the formation of apoptotic bodies, or the DNA cleavage is altered from its original state.

The term “sample,” as used herein, refers to any compositions suspected of containing a Pseudo-ICE binding protein or a ICE-Like binding protein, including but not limited to cell extracts, cDNA expression libraries and recombinant proteins.

As used herein, “promoter” is a segment of DNA or RNA of specific sequence, which controls transcription of the functionally linked DNA or RNA. The specific sequence of the promoter allows for RNA polymerase recognition, binding, and initiation of transcription. The promoter may also include further cis acting or trans acting factors that regulate transcription. The promoter may be constitutive or regulated (inducible) by other factors.

A “yeast two-hybrid screening system” is a system for identifying polypeptide sequences that bind to a predetermined polypeptide sequence through reconstitution of a transcriptional factor in yeast cells. A “transcriptional factor”, as used herein, refers to a protein containing a DNA-binding domain that binds to a regulatory element of a gene and a transcription activation domain that activates the transcription of the gene. Nucleic acids encoding two hybrid proteins, one consisting of a DNA-binding domain of a transcription factor (e.g., Gal 4) fused to the nucleic acid sequence of a known protein and the other consisting of a transcription activation domain of the transcription factor fused to the nucleic acid of a second protein, are constructed and introduced into a yeast host cell. Alternatively, the transcription activation domain of the transcription factor may be fused to the known protein and the DNA-binding domain of the transcription factor may be fused to the second protein. Intermolecular binding between the two fusion proteins reconstitutes the DNA-binding domain with the transcription activation domain, which leads to the transcriptional activation of a reporter gene (e.g., lacZ, His3) operably linked to the DNA binding domain of the transcription factor.

The term “operably linked” refers to functional linkage between a promoter sequence and a structural gene regulated by the promoter sequence. A “structural gene” refers to a DNA sequence that is transcribed into messenger RNA (mRNA), which is then translated into an amino acid sequence. The operably linked promoter controls the expression of the structural gene. The term “expression” generally refers to the transcription of a DNA sequence.

A “host cell” refers to a cell that contains a nucleic acid vector.

An “immunogenic fragment,” as used herein, refers to a polypeptide fragment that can elicit an immune response on injection into a person or animal.

The term “derivitizing” or “derivatizing” refers to standard types of chemical modifications of a compound to produce another structurally related compound typically carried out in the process of compound optimization. The resulting structurally related compound is referred to as a “derivative compound.”

A. Pseudo-ICE and ICE-Like Nucleic Acid Molecules and Uses Thereof

The present invention provides nucleic acid molecules that encode Pseudo-ICE and ICE-Like or fragments of these two polypeptides. The invention discloses nucleic acid sequences encoding representative Pseudo-ICE and ICE-Like polypeptides, such as human Pseudo-ICE and ICE-Like. The nucleotide sequences of human Pseudo-ICE and ICE-Like cDNA were identified by searching the GenBank expressed sequence tag (EST) databases for sequences similar to the prodomain of caspase-1 (residues 1-103) using the tBLASTn program. Two EST clones whose accession numbers are AA070591 and AA046000 corresponding to Pseudo-ICE and ICE-like, respectively, were identified and obtained from the I.M.A.G.E. consortium (Washington University School of Medicine, St Louis, Mo.) and their entire nucleotide sequence was determined by automated sequencing. Based on the sequence of the EST clone AA070591, primers unique to Pseudo-ICE were designed and used in PCR amplification from different human tissues and cell lines. Pseudo-ICE cDNAs were cloned in pcDNA3 (In Vitrogen, Carlsbad, Calif.) and sequenced. Similar procedures were performed to isolated and verify the sequence of ICE-Like cDNA.

Besides human Pseudo-ICE, the invention also includes all nucleic acid sequences that encode Pseudo-ICE or functional fragments thereof that are substantially identical to SEQ ID NO: 1. A Pseudo-ICE that is substantially identical to SEQ ID NO: 1 is at least 80%, 85%, 90%, or 95%, including the percentage of all integer values between 77 and 99, identical to SEQ ID NO: 1. In a preferred embodiment, the nucleic acid sequence is at least 98% or 99% identical to SEQ ID NO: 2. Besides sequence identity, the nucleic acid sequences must also encode polypeptides that have at least one activity associated with Pseudo-ICE of SEQ ID NO: 1.

Likewise, besides human ICE-Like, the invention also includes all nucleic acid sequences that encode ICE-Like or functional fragments thereof that are substantially identical to SEQ ID NO: 3. An ICE-Like that is substantially identical to SEQ ID NO: 3 is at least 70%, 75%, 80%, 85%, 90%, 95%, or 99% identical to SEQ ID NO: 3, including all the integer between 70-99%. Besides sequence identity, the nucleic acid sequences must also encode polypeptides that have at least one activity associated with ICE-Like of SEQ ID NO: 3.

Pseudo-ICE and ICE-Like nucleic acid molecules may be isolated from genomic DNA or cDNA according to practices known in the art (see Sambrook and Russell, Molecular Cloning: A Laboratory Manual, Cold Spring Harbor Press, 2001). Nucleic acid probes corresponding to a region of the Pseudo-ICE or ICE-Like sequences disclosed in the invention may be used to screen either genomic or cDNA libraries. An oligonucleotide probe suitable for screening genomic or cDNA libraries is generally 20-40 bases in length. The oligonucleotide may be synthesized or purchased commercially. The probe may be labeled with a variety of molecules that facilitate detection, such as a radionuclide (e.g., ³²P), an enzymatic label, a protein label, a fluorescent label or biotin.

Genomic and cDNA libraries may be constructed in a variety of suitable vectors including, for example, plasmid, bacteriophage, yeast artificial chromosome and cosmid vectors. Alternatively, libraries may be purchased from a commercial source (e.g., Clontech, Palo Alto, Calif.). Libraries -may contain genomic DNA or cDNA inserts isolated from any species. Nucleotide probes corresponding to the Pseudo-ICE or ICE-Like sequences disclosed in the current application can be used to screen libraries constructed from DNA isolated from other species and, therefore, identify and isolate other Pseudo-ICE or ICE-Like nucleic acid molecules within the scope of the current invention.

Other methods may also be utilized to obtain Pseudo-ICE nucleic acid molecules. One preferred method is to perform polymerase chain reaction (PCR) to amplify a Pseudo-ICE or ICE-Like nucleic acid molecule from cDNA or genomic DNA using oligonucleotide primers corresponding to the 5′ and 3′ ends of Pseudo-ICE or ICE-Like nucleic acid molecules or regions thereof. Detailed methods of PCR cloning may be found in Ausubel, et al., Current Protocols in Molecular Biology, Greene Publishing Associates and Wiley-Interscience, N.Y., 1995, for example.

Another preferred method of obtaining a Pseudo-ICE or ICE-Like nucleic acid molecule is by expression cloning using a polypeptide probe capable of binding a Pseudo-ICE or ICE-Like polypeptide. The probe may comprise Pseudo-ICE or ICE-Like antibodies, or a Pseudo-ICE or ICE-Like binding partner. Methods of expression cloning are described in Sambrook, et al., Molecular Cloning: A Laboratory Manual, Cold Spring Harbor Press, 1989, Ausubel, et al. Current Protocols in Molecular Biology, Greene Publishing Associates and Wiley-Interscience, N.Y., 1995; and Blackwood and Eisenmnan, Methods Enzymology 254:229-240, 1995. Antibody probes suitable for cross-species cloning can include those directed against conserved regions of Pseudo-ICE or ICE-Like polypeptides. Preferably, the antibodies will bind to the denatured Pseudo-ICE or ICE-Like polypeptide.

Polynucleotides of the invention may also be made using the techniques of synthetic chemistry given the sequences disclosed herein. The degeneracy of the genetic code permits alternate nucleotide sequences that encode amino acid sequences presented in SEQ ID NO: 1 or 3. All such nucleotide sequences are within the scope of the present invention.

Isolated genes corresponding to the cDNA sequences disclosed herein are also provided. Methods such as those described above can be used to isolate genes (genomic clones) that correspond to known cDNA sequences. Preferred methods include screening genomic libraries with probes comprising cDNA fragments and PCR amplification of genomic clones from genomic libraries. All polypeptides encoded by the isolated genes are within the scope of the invention. These polypeptides include, but are not limited to, polypeptides encoded by the cDNAs comprising SEQ ID NO: 1 or 3, isoforms of these polypeptides resulting from alternative splicing of the isolated genes, as well as functional fragments thereof.

In certain embodiments, compositions and methods of the invention include ribozymes, antisense RNA and dominant-negative Pseudo-ICE or ICE-Like mutants to decrease levels of functional Pseudo-ICE or ICE-Like polypeptides, respectively. Ribozymes are trans-cleaving catalytic RNA molecules possessing endoribonuclease activity. Ribozymes are specifically designed for a particular target nucleotide sequence. Ribozymes are engineered to cleave an RNA species site-specifically in the background of cellular RNA. The cleavage event renders the mRNA unstable and prevents protein expression. Preparation and usage of ribozymes is well known to the art (see Usman et al., Current Opin. Struct. Biol. 6:527-533, 1996; Long et al., FASEB J. 7:25, 1993; Symons, Ann. Rev. Biochem. 61:641, 1992, and U.S. Pat. No. 5,254,678). The Pseudo-ICE and ICE-Like nucleic acid sequences provided by the invention allow construction of an effective Pseudo-ICE or ICE-Like ribozyme, respectively.

Antisense nucleic acids are designed to specifically bind to RNA, resulting in the formation of RNA-DNA or RNA-RNA hybrids and an arrest in DNA replication, reverse transcription or messenger RNA translation. Antisense polynucleotides based on a selected sequence can specifically interfere with expression of the corresponding gene. Antisense polynucleotides are typically generated within the cell by expression from antisense constructs that contain the antisense strand as the transcribed strand. Antisense production and uses thereof are discussed extensively in the literature and are widely known and available to one skilled in the art (see Argwal et al., Tet. Lett. 28:3539-3542, 1987; Miller et al., J. Am. Chem. Soc. 93:6657-6665, 1971; Stec et al., Tet. Lett. 26:2191-2194, 1985; Moody et al., Nucl. Acids Res. 12:4769-4782, 1989; Eckstein, Trends Biol. Sci. 14:97-100, 1989; Stein In: Oligodeoxynucleotides: Antisense Inhibitors of Gene Expression, Cohen, Ed, Macmillan Press, London, pp. 97-118, 1989; Alama et al., Pharmacol. Res. 36:171-8, 1997; Dean et al., Biochem. Soc. Trans. 24:623-9, 1996; Crooke, Antisense Research and Application, Springer, Heidelberg, 1998; Sandra et al., Am. J. Respir. Cell Mol. Biol. 21:728-37, 1999; U.S. Pat. Nos. 5,168,053; 5,190,931; 5,135,917; 5,087,617; and 5,176,996). Effective Pseudo-ICE or ICE-Like antisense expression vectors may be produced based on the nucleic acid sequences provided by the invention, but typically will include at least 10 nucleotides. In certain embodiments no more than 100 contiguous nucleotides are used, while in other embodiments between 18-60 or between 20-50 contiguous nucleotides are used, including all integer values therebetween.

Nucleic acid sequences encoding Pseudo-ICE or ICE-Like polypeptides may be fused to a variety of heterologous sequences, such as those encoding affinity tags (e.g., GST and His-tag) and those encoding a secretion signal. For instance, when the nucleic acid sequence encoding Pseudo-ICE or ICE-Like is fused to a sequence encoding a secretion signal, the resulting polypeptide is a precursor protein that can be subsequently processed and secreted. The processed Pseudo-ICE or ICE-Like polypeptide may be recovered from the cell lysate, periplasmic space, phloem, or from the growth or fermentation medium. Secretion signals suitable for use are widely available and are well known in the art (e.g., von Heijne, J. Mol. Biol. 184:99-105, 1985).

The Pseudo-ICE or ICE-Like nucleic acid molecules of the subject invention also include variants (including alleles) of the native nucleic acid molecules identified in SEQ ID NO: 2 or 4. Variants of the Pseudo-ICE or ICE-Like nucleic acid molecules provided herein include natural variants (e.g., degenerate forms, polymorphisms, splice variants or mutants) and those produced by genetic engineering. Variants generally have at least 75%, 80%, 85%, 90%, 95%, 98% or 99% (including the percentages of all integer value between 70 and 99) nucleotide identity with SEQ ID NO: 2 or 4. Further, a nucleotide variant will typically be sufficiently similar in sequence to hybridize to the reference sequence under stringent hybridization conditions. For nucleic acid molecules over approximately 50 basepairs, stringent conditions include hybridizing nucleic acid molecules in a solution comprising about 1 M Na⁺ at 25° to 30° C. below the Tm: e.g., 5×SSPE, 0.5% SDS, at 65° C.; and removing insufficiently specific hybridization using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each (see Ausubel et al., Current Protocols in Molecular Biology, Greene Publishing, 1995; Sambrook and Russell, supra).

Nucleic acid sequences which are substantially the same as the nucleic acid sequences encoding Pseudo-ICE or ICE-Like are included within the scope of the invention. Such substantially same sequences may, for example, be substituted with codons optimized for expression in a given host cell such as E. coli. The invention also includes nucleic acid sequences encoding functional domains or fragments of Pseudo-ICE or ICE-Like proteins. Deletions, insertions and/or nucleotide substitutions within a Pseudo-ICE or ICE-Like nucleic acid molecule are also within the scope of the current invention. Such alterations may be introduced by standard methods known in the art such as those described by Ausubel et al., supra. In addition, the invention includes nucleic acids that encode polypeptides that are recognized by antibodies that specifically bind a Pseudo-ICE or ICE-Like polypeptide or fragment thereof.

Exemplary nucleic acids that encode Pseudo-ICE or ICE-Like polypeptides of the present invention have the coding sequences shown in SEQ ID NO: 2 or 4: Polynucleotide molecules of the invention contain less than a whole chromosome and can be single- or double-stranded. Preferably, the polynucleotide molecules are intron-free. Nucleic acid molecules of the invention can comprise at least 11, 15, 18, 21, 30, 33, 42, 54, 60, 66, 72, 84, 90, 100, 120, 140, 160, 180, 200, 220, 240, 260, and all integer values therebetween, contiguous nucleotides of human Pseudo-ICE or ICE-Like gene, the homologues of these genes and the complements of the genes and the homologues as well as degenerate forms.

The Pseudo-ICE or ICE-Like polynucloeotide of the present invention is useful in diagnosing a disease state (e.g., cancer) associated with mutated Pseudo-ICE or ICE-Like polypeptide. For example, the Pseudo-ICE or ICE-Like from a suspected diseased tissue can be sequenced and compared with the Pseudo-ICE or ICE-Like sequence in the normal tissue. Pseudo-ICE or ICE-Like genes, or portions thereof, can be amplified, for example, using nucleotide primers based on the disclosed human Pseudo-ICE or ICE-Like sequence, using the polymerase chain reaction (PCR). The amplified nucleic acid molecules can be sequenced according to any method known in the art such as the dideoxy sequencing method. Nucleotide probes and nucleotides incorporated during sequencing are labeled by a variety of methods, such as radiolabeling, biotinylation, or labeling with fluorescent or chemiluminescent tags, and detected by standard methods known in the art. A difference in the nucleotide sequence of the Pseudo-ICE or ICE-Like from the suspected diseased tissue compared to the Pseudo-ICE or ICE-Like from the normal control sample suggests a role for an encoded Pseudo-ICE or ICE-Like polypeptide in the disease and provides a target for preparing a therapeutic agent.

In addition, the Pseudo-ICE or ICE-Like polynucleotide of the present invention may also be used to diagnose a disease state associated with abnormal Pseudo-ICE or ICE-Like expression levels. Pseudo-ICE or ICE-Like mRNA levels in normal and suspected diseased tissues are compared. PolyA+RNA is isolated from the two tissues as is known in the art. One of skill in the art can then readily determine differences in the size or amount of Pseudo-ICE or ICE-Like -related mRNA transcripts between the two tissues by Northern blot analysis, primer extension, S1 nuclease protection assays, reverse transcription-polymerase chain reaction (RT-PCR), or in situ hybridization using polynucleotide probes corresponding to Pseudo-ICE or ICE-Like or complements thereof. Increased or decreased expression of a Pseudo-ICE- or ICE-Like-related mRNA in a tissue sample suspected of being diseased, compared to the expression of the same Pseudo-ICE or ICE-Like-related mRNA in a normal tissue, suggests that the expressed protein has a role in the disease and also provides a target for preparing a therapeutic agent.

Pseudo-ICE or ICE-Like gene expression can also be examined using polynucleotide arrays. Polynucleotide arrays provide a high throughput technique that can assay large numbers of polynucleotide sequences in a sample. Techniques for constructing arrays and methods of using these arrays are described in U.S. Pat. Nos. 5,593,839; 5,578,832; 5,599,695; 5,556,752; and 5,631,734; which are incorporated by reference.

In addition to diagnostic uses, the nucleic acid molecules of the present invention may also be used to treat various diseases. For instance, because Pseudo-ICE inhibits pro-caspase-1 activation, inhibits IL-1β secretion, and induces or stimulates NF-κB activation, nucleic acid molecules encoding Pseudo-ICE may be introduced to a cell to inhibit apoptosis and/or inflammation or to induce gene expression regulated by NF-κB. The antisense or ribozyme construct of the nucleic acid encoding Pseudo-ICE, on the other hand, may be introduced to a cell to stimulate or activate apoptosis. Likewise, because ICE-Like also inhibits pro-caspase-1 activation and IL-1β secretion, nucleic acid molecules encoding ICE-Like may also be introduced to a cell to inhibit apoptosis and/or inflammation while the antisense or ribozyme construct of the nucleic acids encoding ICE-Like may be used to stimulate apoptosis. The inhibition of apoptosis using the nucleic acid encoding Pseudo-ICE or ICE-Like is beneficial in treating diseases where the inappropriate activation of apoptosis contributes to the pathological states of the diseases such as AIDS, neurodegenerative diseases and ischemic injury. The activation of apoptosis using the antisense or ribozyme construct of the nucleic acid encoding Pseudo-ICE or ICE-Like, on the other hand, is beneficial in treating diseases where the inappropriate loss of apoptosis leads to the pathological accumulation of self reactive lymphocytes, such as various autoimmune diseases.

B. Pseudo-ICE and ICE-Like Polypeptides and Uses Thereof

The present invention includes Pseudo-ICE and ICE-Like polypeptide sequences or functional fragments thereof that are identical or substantially identical to human Pseudo-ICE and ICE-Like as set forth in SEQ ID NOS: 1 and 3, respectively. Such Pseudo-ICE and ICE-Like polypeptides include native Pseudo-ICE and ICE-Like polypeptides that are naturally present in organisms, non-native Pseudo-ICE and ICE-Like polypeptides, and Pseudo-ICE and ICE-Like polypeptide fusion proteins. Native Pseudo-ICE and ICE-Like polypeptide include all the variants resulting from polymorphism, alternative mRNA splicing/transcription, or differential polypeptide processing. Non-native Pseudo-ICE and ICE-Like may contain an amino acid deletion, insertion, or substitution of native Pseudo-ICE and ICE-Like, respectively. Pseudo-ICE and ICE-Like fusion proteins can be Pseudo-ICE and ICE-Like polypeptides or functional fragments thereof fused with any other known proteins or portions thereof.

As defined above, Pseudo-ICE polypeptides of the present invention include polypeptides of any origin that are substantially homologous to the human Pseudo-ICE polypeptide as set forth in SEQ ID NO: 1 and that has at least one function of the polypeptide of SEQ ID NO: 1. More specifically, a Pseudo-ICE polypeptide of the present invention generally has an amino acid sequence at least 80%, 85%, 90%, 95%, 99% (including the percentages of all integer values between 80 and 90) identical to a human Pseudo-ICE (SEQ ID NO: 1). In addition to the sequence identity to SEQ ID NO: 1, the Pseudo-ICE of the present invention is also able to perform at least one of the following fumctions: to interact with pro-caspase-1, to interact with RICK, to inhibit IL-1β secretion induced by an inflammatory stimulus, and to induce NF-κB activation. Methods for determining whether a particular peptide has the above functions are known in the art. Examples of such methods are described infra (e.g., in Examples).

Likewise, ICE-Like polypeptides of the present invention include polypeptides of any origin that are substantially homologous to the human ICE-Like polypeptide as set forth in SEQ ID NO: 3 and that has at least one function associated with the polypeptide of SEQ ID NO: 3. More specifically, an ICE-Like polypeptide of the present invention generally has an amino acid sequence at least 80%, 85%, 90%, 95%, 99% (including the percentages of all integer values between 80-90) identical to a human Pseudo-ICE (SEQ ID NO: 3). In addition to the sequence identity to SEQ ID NO: 1, the ICE-Like of the present invention also has the ability to interact with pro-caspase-1 and/or to inhibit IL-1β secretion induced by an inflammatory stimulus. Methods for determining whether a particular peptide has the above functions are known in the art. Examples of such methods are described infra (e.g, in Examples).

Also as defined above, a functional fragment of Pseudo-ICE or ICE-Like of the present invention includes a fragment of a full length Pseudo-ICE or ICE-Like polypeptide of the present invention that retains at least one functional activity associated with the full-length Pseudo-ICE or ICE-Like, respectively. The functional fragment is typically large enough to function as a binding domain for an antibody or for another polypeptide and usually have at least 10, 35, 40, 50, 60 (including all integer values between 10 and 60) amino acids that are substantially homologous to a portion of SEQ ID NO: 1 or3.

For non-native Pseudo-ICE or ICE-Like, guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting variant. Whether an amino acid change results in a functional protein or polypeptide can readily be determined by testing the altered protein or polypeptide in a functional assay, for example, as disclosed in U.S. Pat. No. 5,654,173 and described in detail below.

A conservative amino acid change involves substitution of one amino acid for another amino acid of a family of amino acids with structurally related side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and unchareged polar (glycine, asparagines, glutamine, cysteine, serine, threonine, tyrosine) amino acids. Phenylalanie, tryptophan, and tyrosine are sometimes classified as aromatic amino acids. Non-naturally occurring amino acids can also be used to form protein variants of the invention.

Pseudo-ICE or ICE-Like fusion proteins of the invention include polypeptides comprising Pseudo-ICE or ICE-Like polypeptides or fragments thereof fused to amino acid sequences comprising one or more heterologous polypeptides. Such heterologous polypeptides may correspond to naturally occurring polypeptides of any source or may be recombinantly engineered amino acid sequences. Fusion proteins are useful for purification, generating antibodies against amino acid sequences, and for use in various assay systems. For example, fusion proteins can be used to identify proteins which interact with a protein of the invention or which interfere with its biological function. Fusion proteins comprising a signal sequence and/or a transmembrane domain of one or more of the disclosed proteins can be used to target other protein domains to cellular locations in which the domains are not normally found, such as bound to a cellular membrane or secreted extracellularly.

The polypeptides that Pseudo-ICE or ICE-Like polypeptides or functional fragments thereof fused to can be full-length proteins or polypeptide fragments. Proteins commonly used in fusion protein construction include β-galactosidase, β-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions.

An isolated Pseudo-ICE or ICE-Like polypeptide or functional fragment thereof can be obtained by a variety of methods known in the art. For example, a Pseudo-ICE or ICE-Like polypeptide can be isolated by biochemical methods such as affinity chromatography. Affinity matrices that can be used for Pseudo-ICE or ICE-Like isolation can be a solid phase having attached thereto anti-Pseudo-ICE or anti-ICE-Like monoclonal or polyclonal antibodies prepared against a Pseudo-ICE or ICE-Like polypeptide or a functional fragment thereof comprising a Pseudo-ICE or ICE-Like epitope. Alternatively, polypeptides known to bind Pseudo-ICE or ICE-Like (e.g., caspase-1) can be used as affinity matrices to isolate a Pseudo-ICE or ICE-Like polypeptide or functional fragment thereof.

Other biochemical methods for isolating a Pseudo-ICE or ICE-Like polypeptide or functional fragment thereof include preparative gel electrophoresis, gel filtration, affinity chromatography, ion exchange and reversed phase chromatography, chromatofocusing, isoelectric focusing and sucrose or glycerol density gradients (Deutscher, Methods in Enzymology: Guide to Protein Purification, Vol. 182, Academic Press, Inc., San Diego, Chapter 38, 1990; Balch et al., Methods in Enzymology, Vol. 257, Academic Press, Inc., San Diego, Chapter 8, 1995). For example, a Pseudo-ICE or ICE-Like polypeptide or functional fragment thereof can be isolated by preparative polyacrylamide gel electrophoresis and elution by diffusion or electroelution (Deutscher, supra, Chapter 33, 1990). Continuous elution gel electrophoresis using a system such as the Model 491 Prep Cell (BioRad, Hercules, Calif.) can be used to isolate a Pseudo-ICE or ICE-Like polypeptide or functional fragment thereof. If desired, continuous elution gel electrophoresis can be combined with further purification steps such as liquid phase preparative isoelectric focusing using, for example, the Rotofor system (BioRad).

A Pseudo-ICE or ICE-Like polypeptide or functional fragment thereof also can be produced by chemical synthesis, for example, by the solid phase peptide synthesis method (Merrifield et al., J. Am. Chem. Soc. 85:2149, 1964). Standard solution methods well known in the art also can be used to synthesize a Pseudo-ICE or ICE-Like polypeptide or functional fragment thereof (Bodanszky, Principles of Peptide Synthesis, Springer-Verlag, Berlin, 1984; Bodanszky, Peptide Chemistry, Springer-Verlag, Berlin, 1993). A newly synthesized Pseudo-ICE or ICE-Like polypeptide or functional fragment thereof can be isolated, for example, by high performance liquid chromatography and can be characterized using mass spectrometry or amino acid sequence analysis.

In addition, Pseudo-ICE or ICE-Like or analogues thereof can be designed to have increased stability in vivo or in vitro or higher or lower affinity of binding to a pro-domain of a caspase-1 protein by incorporating, for example, (D)-amino acids into a Pseudo-ICE or ICE-Like peptide or by chemically modifying reactive amino acid side chains or the amino or carboxy terminus of a peptide. For example, a reactive amino group in a peptide can be rendered less reactive by acetylation. Furthermore, a modification such as acetylation changes a hydrophilic group to a hydrophobic group, which can be advantageous, when it is desirable to prepare a Pseudo-ICE or ICE-Like peptide that can readily traverse a cell membrane.

A Pseudo-ICE or ICE-Like polypeptide or functional fragment thereof can also be produced by recombinant DNA methods. Nucleic acids encoding Pseudo-ICE or ICE-Like polypeptides or functional fragments thereof provided by the invention can be cloned into an appropriate vector for expression. Such a vector is commercially available or can be constructed by those skilled in the art and contains expression elements necessary for the transcription, translation, regulation, and, if desired, sorting of the Pseudo-ICE or ICE-Like polypeptide or functional fragment thereof. The selected vector can also be used in a procaryotic or eukaryotic host system, as appropriate, provided the expression and regulatory elements are of compatible origin. A recombinant Pseudo-ICE or ICE-Like polypeptide or functional fragment thereof produced in a host cell or secreted from the cell can be isolated using, for example, affinity chromotography with an anti-Pseudo-ICE or anti-ICE-Like antibody, ionic exchange chromatography, HPLC, size exclusion chromatography, ammonium sulfate crystallization, electrofocusing, or preparative gel electrophoresis (see generally Ausubel et al., supra; Sambrook et al., supra). An isolated purified protein is generally evidenced as a single band on an SDS-PAGE gel stained with Coomassie Blue.

Pseudo-ICE or ICE-Like fusion polypeptides of the invention can be made by covalently linking two protein segments or by standard procedures in the art of molecular biology. For example, recombinant DNA methods can be used to prepare fusion proteins by making a DNA construct which comprises coding sequences selected from SEQ ID NOS: 2 or 4 in proper reading frame with nucleotides encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

As discussed above, both Pseudo-ICE and ICE-Like are inhibitors of pro-caspase-1 activation and IL-1β secretion, and thus inhibitors of apoptosis and inflammation. Dysregulated apoptosis is a part of many diseases and disorders, including cancer, autoimmunity, and neurodegenerative disorders. Controlling apoptosis is a way to counteract or treat diseases. Treatment refers to a lessening or amelioration of the disease, symptoms, or other effects of the disease. Patients suitable for treatment with the compositions described herein are identified by well-known hallmarks of the particular diseases.

As noted above, pharmaceutical compositions also are provided by this invention. These compositions may contain Pseudo-ICE or ICE-Like polypeptide or a functional fragment thereof (or nucleic acid molecules comprising a nucleotide sequence encoding Pseudo-ICE or ICE-Like polypeptide or functional fragment thereof) along with a pharmaceutically or physiologically acceptable carrier, excipients or diluents. Generally, such carriers should be nontoxic to recipients at the dosages and concentrations employed. Ordinarily, the preparation of such compositions entails combining the therapeutic agent with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents.

In addition, the pharmaceutical compositions of the present invention may be prepared for administration by a variety of different routes, including for example intraarticularly, intracranially, intradermally, intrahepatically, intramuscularly, intraocularly, intraperitoneally, intrathecally, intravenously, subcutaneously or even directly into a tumor. In addition, pharmaceutical compositions of the present invention may be placed within containers, along with packaging material that provides instructions regarding the use of such pharmaceutical compositions. Generally, such instructions will include a tangible expression describing the reagent concentration, as well as within certain embodiments, relative amounts of excipient ingredients or diluents (e.g, water, saline or PBS) that may be necessary to reconstitute the pharmaceutical composition. Pharmaceutical compositions are useful for both diagnostic and therapeutic purposes.

The compositions may be administered in a delivery vehicle. For example, the composition can be encapsulated in a liposome (see, e.g., WO 96/10585; WO 95/35094), complexed with lipids, encapsulated in slow-release or sustained release vehicles, such as poly-galactide, and the like. Within other embodiments, compositions may be prepared as a lyophilizate, utilizing appropriate excipients to provide stability.

The level of therapeutic in serum and other tissues after administration can be monitored by various well-established techniques such as chromatographic or antibody based, such as ELISA, assays.

Pharmaceutical compositions of the present invention may be administered in a manner appropriate to the disease to be treated (or prevented). The quantity and frequency of administration will be determined by such factors as the condition of the patient, and the type and severity of the patient's disease. Dosages may be determined most accurately during clinical trials. Patients may be monitored for therapeutic effectiveness by appropriate technology, including signs of clinical exacerbation, imaging and the like.

C. Vectors Host Cells and Means of Expressing and Producing Proteins

The present invention encompasses vectors comprising regulatory elements linked to Pseudo-ICE or ICE-Like nucleic acid sequences. Such vectors may be used, for example, in the propagation and maintenance of Pseudo-ICE or ICE-Like nucleic acid molecules or the expression and production of Pseudo-ICE or ICE-Like polypeptides and nucleic acid molecules. Vectors may include, but are not limited to, plasmids, episomes, baculovirus, retrovirus, lentivirus, adenovirus, and parvovirus including adeno-associated virus.

Pseudo-ICE or ICE-Like may be expressed in a variety of host organisms. In certain embodiments, Pseudo-ICE or ICE-Like is produced mammalian cells, such as CHO, COS-7, or 293 cells. Other suitable host organisms include bacterial species (e.g., E. coli and Bacillus) other eukaryotes, such as yeast (e.g., Saccharomyces cerevisiae), plant cells and insect cells (e.g., Sf9). Vectors for these hosts are well known in the art.

A DNA sequence that encodes Pseudo-ICE or ICE-Like is introduced into an expression vector appropriate for the host. The sequence is derived from an existing clone or synthesized. As described herein, a fragment of the coding region may be used. A preferred means of synthesis is amplification of the gene from cDNA, genomic DNA, or a recombinant clone using a set of primers that flank the coding region or the desired portion of the protein. Restriction sites are typically incorporated into the primer sequences and are chosen with regard to the cloning site of the vector. If necessary, translation initiation and termination codons can be engineered into the primer sequences. The sequence of Pseudo-ICE or ICE-Like can be codon-optimized for expression in a particular host. For example, a Pseudo-ICE or ICE-Like isolated from a human cell that is expressed in a fungal host, such as yeast, can be altered in nucleotide sequence to use codons preferred in yeast. Further, it may be beneficial to insert a traditional AUG initiation codon at the CUG initiation positions to maximize expression, or to place an optimized translation initiation site upstream of the CUG initiation codon. Accordingly, such codon-optimization may be accomplished by methods such as splice overlap extension, site-directed mutagenesis, automated synthesis, and the like.

At minimum, the vector must contain a promoter sequence. As used herein, a “promoter” refers to a nucleotide sequence that contains elements that direct the transcription of a linked gene. At minimum, a promoter contains an RNA polymerase binding site. More typically, in eukaryotes, promoter sequences contain binding sites for other transcriptional factors that control the rate and timing of gene expression. Such sites include TATA box, CAAT box, POU box, AP1 binding site, and the like. Promoter regions may also contain enhancer elements. When a promoter is linked to a gene so as to enable transcription of the gene, it is “operatively linked”.

Typical regulatory elements within vectors include a promoter sequence that contains elements that direct transcription of a linked gene and a transcription termination sequence. The promoter may be in the form of a promoter that is naturally associated with the gene of interest. Alternatively, the nucleic acid may be under control of a heterologous promoter not normally associated with the gene. For example, tissue specific promoter/enhancer elements may be used to direct expression of the transferred nucleic acid in repair cells. In certain instances, the promoter elements may drive constitutive or inducible expression of the nucleic acid of interest. Mammalian promoters may be used, as well as viral promoters capable of driving expression in mammalian cells. Examples of other regulatory elements that may be present include secretion signal sequences, origins of replication, selectable markers, recombinase sequences, enhancer elements, nuclear localization sequences (NLS) and matrix association regions (MARS).

The expression vectors used herein include a promoter designed for expression of the proteins in a host cell (e.g., bacterial). Suitable promoters are widely available and are well known in the art. Inducible or constitutive promoters are preferred. Such promoters for expression in bacteria include promoters from the T7 phage and other phages, such as T3, T5, and SP6, and the trp, lpp, and lac operons. Hybrid promoters (see U.S. Pat. No. 4,551,433), such as tac and trc, may also be used. Promoters for expression in eukaryotic cells include the P10 or polyhedron gene promoter of baculovirus/insect cell expression systems (see, e.g., U.S. Pat. Nos. 5,243,041, 5,242,687, 5,266,317, 4,745,051, and 5,169,784), MMTV LTR, CMV IE promoter, RSV LTR, SV40, metallothionein promoter (see, e.g., U.S. Pat. No. 4,870,009) and the like.

The promoter controlling transcription of Pseudo-ICE or ICE-Like may itself be controlled by a repressor. In some systems, the promoter can be de-repressed by altering the physiological conditions of the cell, for example, by the addition of a molecule that competitively binds the repressor, or by altering the temperature of the growth media. Preferred repressor proteins include, but are not limited to, the E coli lacI repressor responsive to IPTG induction, the temperature sensitive λcI857 repressor, and the like. The E. coli lacI repressor is preferred.

In other preferred embodiments, the vector also includes a transcription terminator sequence. A “transcription terminator region” has either a sequence that provides a signal that terminates transcription by the polymerase that recognizes the selected promoter and/or a signal sequence for polyadenylation.

Preferably, the vector is capable of replication in the host cells. Thus, when the host cell is a bacterium, the vector preferably contains a bacterial origin of replication. Preferred bacterial origins of replication include the f1-ori and col E1 origins of replication, especially the ori derived from pUC plasmids. In yeast, ARS or CEN sequences can be used to assure replication. A well-used system in mammalian cells is SV40 ori.

The plasmids also preferably include at least one selectable marker that is functional in the host. A selectable marker gene includes any gene that confers a phenotype on the host that allows transformed cells to be identified and selectively grown. Suitable selectable marker genes for bacterial hosts include the ampicillin resistance gene (Amp^{r}), tetracycline resistance gene (Tc^{r}) and the kanamycin resistance gene (Kan^{r}). The kanamycin resistance gene is presently preferred. Suitable markers for eukaryotes usually require a complementary deficiency in the host (e.g, thymidine kinase (tk) in tk-hosts). However, drug markers are also available (e.g., G418 resistance and hygromycin resistance).

The sequences of nucleotides encoding Pseudo-ICE or ICE-Like may also include a secretion signal, whereby the resulting peptide is a precursor protein processed and secreted. The resulting processed protein may be recovered from the periplasmic space, the growth medium, phloem, etc. Secretion signals suitable for use are widely available and are well known in the art (von Heijne, J. Mol. Biol. 184:99-105, 1985). Prokaryotic and eukaryotic secretion signals that are functional in E. coli (or other host) may be employed. The presently preferred secretion signals include, but are not limited to, those encoded by the following E. Coli genes: pelB (Lei et al., J. Bacteriol. 169:4379, 1987), phoA, ompA, ompT, ompF, ompC, beta-lactamase, and alkaline phosphatase.

One skilled in the art appreciates that there are a wide variety of suitable vectors for expression in bacterial cells and which are readily obtainable. Vectors such as the pET series (Novagen, Madison, Wis.), the tac and trc series (Pharmacia, Uppsala, Sweden), pTTQ18 (Amersham International plc, England), pACYC 177, pGEX series, and the like are suitable for expression of Pseudo-ICE or ICE-Like. Baculovirus vectors, such as pBlueBac (see, e.g., U.S. Pat. Nos. 5,278,050, 5,244,805, 5,243,041, 5,242,687, 5,266,317, 4,745,051, and 5,169,784; available from Invitrogen, San Diego) may be used for expression in insect cells, such as Spodoptera frugiperda sf9 cells (see U.S. Pat. No. 4,745,051). The choice of a bacterial host for the expression of Pseudo-ICE or ICE-Like is dictated in part by the vector. Commercially available vectors are paired with suitable hosts.

A wide variety of suitable vectors for expression in eukaryotic cells are also available. Such vectors include pCMVLacI, pXT1 (Stratagene Cloning Systems, La Jolla, Calif.); pCDNA series, pREP series, pEBVHis (Invitrogen, Carlsbad, Calif.). In certain embodiments, Pseudo-ICE or ICE-Like gene is cloned into a gene targeting vector, such as pMC1neo, a pOG series vector (Stratagene Cloning Systems).

The introduction of a vector into various cells, such as bacterial, yeast, insect, mammalian, and plant cells, are well known. For example, a vector can be transformed into a bacterial cell by heat shock or electroporation. Transformation of a yeast cell with a vector may also be carried out by electroporation. Methods for introduction of vectors into animal cells include calcium phosphate precipitation, electroporation, dextran-mediated transfection, liposome encapsulation, nucleus microinjection, and viral or phage infection. The introduction of heterologous nucleic acid sequences into plant cells can be achieved by particle bombardment, electroporation, microinjection, and Agrobacterium-mediated gene insertion (for reviews of such techniques, see, for example, Weissbach & Weissbach, Methods for Plant Molecular Biology, Academic Press, N.Y., Section VHI, pp. 421-463; 1988; Grierson & Corey, Plant Molecular Biology, 2d Ed., Blackie, London, Ch. 7-9, 1988, and Horsch et al., Science 227:1229, 1985; and Gene Transfer to Plants, eds. Potrykus. Springer Verlaa, 1995, all incorporated herein by reference).

D. Pseudo-ICE and ICE-Like Antibodies and Uses Thereof

The present invention also provides antibodies that specifically bind to a Pseudo-ICE or ICE-Like polypeptide. An antibody is said to “specifically bind” to Pseudo-ICE or ICE-Like if it binds to Pseudo-ICE or ICE-Like at a detectable level, but does not bind detectably to peptides containing an unrelated sequence. In certain embodiments, the dissociation constant of the interaction between an antibody molecule and a Pseudo-ICE or ICE-Like polypeptide or a functional fragment thereof is at most 10⁻⁷ M. In other embodiments, the dissociation constant is at most 10^{—8} M. Preferred binding molecules include antibodies, which may be, for example, polyclonal, monoclonal, single chain, chimeric, or CDR-grafted antibodies, or fragments thereof, such as proteolytically generated or recombinantly produced F(ab′)₂, Fab, Fv, and Fd fragments. Certain preferred antibodies are those antibodies that inhibit or block one of the Pseudo-ICE or ICE-Like activities within an in vitro assay, as described herein. Binding properties of the antibody to Pseudo-ICE or ICE-Like may generally be assessed using immunodetection methods including, for example, an enzyme-linked immunosorbent assay (ELISA), immunoprecipitation, and immunoblotting, which may be readily performed by those having ordinary skill in the art.

Methods well known in the art may be used to generate antibodies, polyclonal antisera, or monoclonal antibodies that are specific for Pseudo-ICE or ICE-Like. Antibodies also may be produced as genetically engineered immunoglobulins (Ig) or Ig fragments designed to have desirable properties. For example, by way of illustration and not limitation, antibodies may include a recombinant IgG that is a chimeric fusion protein having at least one variable (V) region domain from a first mammalian species and at least one constant region domain from a second distinct mammalian species. Most commonly, a chimeric antibody has murine variable region sequences and human constant region sequences. Such a murine/human chimeric immunoglobulin may be “humanized” by grafting the complementarity determining regions (CDRs), which confer binding specificity for an antigen, derived from a murine antibody into human-derived V region framework regions and human-derived constant regions. Fragments of these molecules may be generated by proteolytic digestion, or optionally, by proteolytic digestion followed by mild reduction of disulfide bonds and alkylation, or by recombinant genetic engineering techniques.

Antibodies may generally be prepared by any of a variety of techniques known to those having ordinary skill in the art. See, e.g., Harlow et al., Antibodies: A Laboratory Manual, Cold Spring Harbor Laboratory, 1988. In one such technique, an animal is immunized with a Pseudo-ICE or ICE-Like polypeptide or a functional fragment of the Pseudo-ICE or ICE-Like polypeptide as antigen to generate polyclonal antisera. Suitable animals include rabbits, sheep, goats, pigs, cattle, and may include smaller mammalian species, such as mice, rats, and hamsters.

An immunogen may be comprised of cells expressing a Pseudo-ICE or ICE-Like polypeptide or portions thereof, purified or partially purified Pseudo-ICE or ICE-Like polypeptides or functional fragments thereof. Pseudo-ICE or ICE-Like functional fragments may be generated by proteolytic cleavage or may be chemically synthesized. The present disclosure provides examples of nucleic acid sequences encoding Pseudo-ICE or ICE-Like polypeptides and functional fragments thereof, such that those skilled in the art may routinely prepare them for use as immunogens. Peptides for immunization may also be selected by analyzing the primary, secondary, and tertiary structure of Pseudo-ICE or ICE-Like according to methods known to those skilled in the art in order to determine amino acid sequences more likely to generate an antigenic response in a host animal. See, e.g., Novotny, Mol. Immunol. 28:201-207, 1991; Berzoksky, Science 229:932-40, 1985.

Monoclonal antibodies that specifically bind to Pseudo-ICE or ICE-Like polypeptides or functional fragments thereof may be prepared, for example, using the technique of Kohler and Milstein (Nature, 256:495-497, 1975; Eur. J. Immunol. 6:511-519, 1976) and improvements thereto. Hybridomas, which are immortal eukaryotic cell lines, may be generated that produce antibodies having the desired specificity to a Pseudo-ICE or ICE-Like polypeptide or a fragment thereof. An animal—for example, a rat, hamster, or preferably mouse—is immunized with Pseudo-ICE or ICE-Like immunogen prepared as described above. Lymphoid cells, most commonly, spleen cells, obtained from an immunized animal may be immortalized by fusion with a drug-sensitized myeloma cell fusion partner, preferably one that is syngeneic with the immunized animal. The spleen cells and myeloma cells may be combined for a few minutes with a membrane fusion-promoting agent, such as polyethylene glycol or a nonionic detergent, and then plated at low density on a selective medium that supports the growth of hybridoma cells, but not myeloma cells. A preferred selection media is HAT (hypoxanthine, aminopterin, thymidine). After a sufficient time, usually about 1 to 2 weeks, colonies of cells are observed. Single colonies are isolated, and antibodies produced by the cells may be tested for binding activity to the Pseudo-ICE or ICE-Like polypeptide or variant or fragment thereof. Hybridomas producing antibody with high affinity and specificity for the Pseudo-ICE or ICE-Like antigen are preferred. Hybridomas that produce monoclonal antibodies that specifically bind to a Pseudo-ICE or ICE-Like polypeptide or variant or fragment thereof are contemplated by the present invention.

Within certain embodiments, the use of antigen-binding fragments of antibodies may be preferred. Such fragments include single-chain Fv, Fab fragments or F(ab′)₂ fragments, which may be prepared by proteolytic digestion with papain or pepsin, respectively. The antigen binding fragments may be separated from the Fc fragments by affinity chromatography, for example, using immobilized protein A or immobilized Pseudo-ICE or ICE-Like polypeptide or a functional fragment thereof. An alternative method to generate Fab fragments includes mild reduction of F(ab′)₂ fragments followed by alkylation. See, e.g., Weir, Handbook of Experimental Immunology, 1986, Blackwell Scientific, Boston, 1986.

An additional method for selecting antibodies that specifically bind to a Pseudo-ICE or ICE-Like polypeptide or a functional fragment thereof is by phage display. See, e.g., Winter et al., Annul. Rev. Immunol. 12:433-55, 1994; Burton et al., Adv. Immunol. 57:191-280, 1994. Human or murine immunoglobulin variable region gene combinatorial libraries may be created in phage vectors that can be screened to select Ig fragments (Fab, Fv, sFv, or multimers thereof) that bind specifically to a Pseudo-ICE or ICE-Like polypeptide or a functional fragment thereof. See, e.g., U.S. Pat. No. 5,223,409; Huse et al., Science 246:1275-81, 1989; Kang et al., Proc. Natl. Acad. Sci. USA 88:4363-66, 1991; Hoogenboom et al., J. Molec. Bio. 227:381-388, 1992; Schlebusch et al., Hybridoma 16:47-52, 1997 and references cited therein. For example, polynucleotide sequences encoding Ig variable region fragments may be inserted into the genome of a filamentous bacteriophage, such as M13 or a variant thereof, in frame with the sequence encoding a phage coat protein, for example, gene III or gene VIII of M13, to create a fusion protein. A fusion protein may be a fusion of the coat protein with either the light chain variable region domain or the heavy chain variable region domain. Upon the identification of the variable region domain that specifically binds to a Pseudo-ICE or ICE-Like polypeptide or a functional fragment of the Psuedo-ICE or ICE-Like polypeptide, nucleic acid molecules that encode antibodies or antigen-binding fragments of antibodies may be generated. Expression of such molecules in a host cell may decrease the level of functional Pseudo-ICE or ICE-Like polypeptides, thus moderate apoptosis in the cell.

Antibodies may be assayed for immunoreactivity against Pseudo-ICE or ICE-Like by any of a number of methods, including western blot, enzyme-linked immuno-sorbent assays (ELISA), countercurrent immuno-electrophoresis, radioimmunoassays, dot blot assays, sandwich assays, inhibition or competition assays, or immunoprecipitation (see U.S. Pat. Nos. 4,376,110 and 4,486,530; see also Antibodies: A Laboratory Manual, Harlow and Lane (eds.), Cold Spring Harbor Laboratory Press, 1988). Techniques for purifying antibodies are those available in the art. In certain embodiments, antibodies are purified by passing the antibodies over an affinity column that contains bound amino acid sequences of the present invention. Bound antibody is then eluted. Other purification techniques include, but are not limited to HPLC or RP-HPLC, or purification on protein A or protein G columns.

Antibodies that bind Pseudo-ICE or ICE-Like polypeptides can be used for diagnostic purposes. Any method known in the art can be used to compare Pseudo-ICE or ICE-Like proteins from normal control samples and suspected diseased samples. The size of the proteins in the two samples can be compared, for example, using antibodies against Pseudo-ICE or ICE-Like polypeptides to detect Pseudo-ICE or ICE-Like polypeptides, respectively, by Western blot. Alterations in the size of the Pseudo-ICE or ICE-Like protein in a tissue suspected of being diseased compared with the level in a normal control sample indicate the protein is abnormal, possibly due to truncation, deletion, or altered post-translation modification. Size alterations are indicative that Pseudo-ICE or ICE-Like has a role in the disease. Other changes, such as protein expression levels and subcellular localization can also be detected immunologically, for example, by using antibodies directed against polypeptides encoded by Pseudo-ICE or ICE-Like for Western blot or immuno-fluorescence. A higher or lower level of Pseudo-ICE or ICE-Like protein in a tissue from a subject with a disease compared with the level in a normal control sample is indicative that Pseudo-ICE or ICE-Like has a role in the disease. Similarly, changes in the subcellular localization of Pseudo-ICE or ICE-Like protein also indicate that Pseudo-ICE or ICE-Like has a role in the disease.

In addition, antibodies specific to Pseudo-ICE or ICE-Like may also be used to treat diseases associated with inappropriate loss of apoptosis. Because Pseudo-ICE or ICE-Like binds to caspase-1 and inhibits the activation of caspase-1, antibodies specific to Pseudo-ICE or ICE-Like may bind Pseudo-ICE or ICE-Like and thus prevent Pseudo-ICE or ICE-Like from inhibiting the activation of caspase-1, resulting in stimulating apoptosis. Accordingly, antibodies specific to Pseudo-ICE or ICE-Like or nucleic acid molecules encoding these antibodies may be used as active components of pharmaceutical compositions for treating diseases such as autoimmune diseases, which are associated with loss of apoptosis.

E. Methods of Identifying Effectors of Pseudo-ICE or ICE-Like Mediated Processes

The present invention also provides methods for identifying effectors of Pseudo-ICE or ICE-Like mediated processes. The term “effector,” as used herein, includes any molecule that has an effect on Pseudo-ICE or ICE-Like mediated processes and can be an inhibitor or an enhancer of these processes. Pseudo-ICE or ICE-Like mediated processes include, but are not limited to, pro-capsase-1 oligomerization, pro-caspase-1 activation, IL-1β secretion, and NF-κB activation.

The effector may act by preventing expression of Pseudo-ICE or ICE-Like, by preventing binding of Pseudo-ICE or ICE-Like to partner proteins, by causing dissociation of the bound proteins, or by some other mechanism. Furthermore, the effector may act directly or indirectly. In preferred embodiments, effectors interfere with the binding of Pseudo-ICE or ICE-Like protein to a pro-caspase-1 or a fragment thereof (e.g., a pro-domain of a pro-caspase-1). Effectors may also interfere with the binding of Pseudo-ICE to RICK or a fragment thereof. In other preferred embodiments, the effectors are small molecules. Effectors should have a minimum of side effects and are preferably non-toxic. Effectors that can penetrate cells are preferred.

Candidate effectors may be isolated or procured from a variety of sources, such as bacteria, fungi, plants, parasites, libraries of chemicals, random peptides or the like. Candidate effectors may also be variants of Pseudo-ICE or ICE-Like or functional fragment thereof, antisense to nucleic acids encoding Pseudo-ICE or ICE-Like, inhibitors of promoter activity of Pseudo-ICE or ICE-Like, and the like. Effectors may also be rationally designed, based on the protein structure determined from X-ray crystallography (see, Livnah et al., Science 273:464, 1996). In addition, effectors may be also further derivatized using conventional chemistry to generate other structurally related effectors with more desirable features (e.g., higher affinity to Pseudo-ICE or ICE-Like than original effectors, and higher activities in disrupting binding between Pseudo-ICE or ICE-Like and a binding partner thereof).

The effectors of the present invention may find utilities in treating diseases associated with abnormal processes mediated by Pseudo-ICE or ICE-Like as described above. Such diseases include, but are not limited to, chronic and acute inflammatory, autoimmune or neurodegenerative diseases.

The present invention provides methods for identifying effectors of Pseudo-ICE or ICE-Like mediated processes. Methods will vary according to the type of effector and nature of the activity that is being altered. Such methods comprise protein binding assays, measurement of pro-caspase-1 processing, caspase-1 activity, IL-1β secretion, NF-κB activation or the like.

The effector of the present invention may be a polypeptide that specifically binds to Pseudo-ICE or ICE-Like. Such an effector may be identifying by contacting a sample with a Pseudo-ICE or ICE-Like polypeptide or a functional fragment thereof and detecting the formation of complex between the Pseudo-ICE or ICE-Like polypeptide or functional fragment thereof and the binding polypeptide. The sample can be any composition suspected of containing a Pseudo-ICE or ICE-Like binding protein, including but not limited to cell extracts, cDNA expression libraries and recombinant proteins. The Pseudo-ICE or ICE-Like polypeptide or its functional fragment in the binding reaction can be a native polypeptide or a recombinant polypeptide protein. Preferably, the Pseudo-ICE or ICE-Like polypeptide or its functional fragment is covalently bound to a detectable moiety, such as a reporter molecule, a radionuclide, or a polypeptide that facilitates the purification of the polypeptide, such as MBP and His tag. In certain embodiments, either before or after contacting with a sample containing an apoptotic pathway protein, a Pseudo-ICE or ICE-Like polypeptide or its functional fragment is attached to a solid support via, e.g., its specific antibodies, or the polypeptide fused to it. The invention also includes the method of detecting or identifying Pseudo-ICE or ICE-Like associated protein where the interaction between the Pseudo-ICE or ICE-Like polypeptide and the binding protein occurs in vivo. The resulting complex of Pseudo-ICE or ICE-Like polypeptide and the binding protein is then purified and its components other than the Pseudo-ICE or ICE-Like polypeptide identified by any known protein purification and detection method. Several specific embodiments are described in more detail below. Additional methods such as detecting protein interactions using green fluorescence protein and fluorescence resonance energy transfer and analyzing interacting proteins with surface plasmon resonance spectroscopy using BIAcore, may be found in Sambrook and Russell, supra.

In Vitro Protein-Protein Interaction

Any in vitro protein-protein interaction assays can be used to detect a protein that interacts with a Pseudo-ICE or ICE-Like polypeptide. In one example, a fusion protein is constructed comprising a Pseudo-ICE or ICE-Like polypeptide or a functional fragment thereof and a tag peptide sequence (e.g., GST). The GST-fusion protein is then purified using glutathione-Sepharose beads (see, Kaelin et al., Cell 64:521, 1991). The bead-bound, purified fusion protein is then incubated with a sample, such as a plant cell extract (usually matabolically radio-labeled). Proteins that are not bound to the beads are washed away and those bound eluted. The bound proteins may be further fractionated by gel electrophoresis and detected by protein dyes or the radioactive label on the protein. They may be then used for raising antibodies, amino acid sequence analysis, and other in vitro analyses. Clones encoding the bound proteins may be isolated by any standard methods, including immunoscreening of an expression library, probe hybridization where the probe is based on partial amino acid sequences of the bound proteins.

In another example, a Pseudo-ICE or ICE-Like polypeptide (or a functional fragment thereof) is first immobilized to a solid support (e.g., an ELISA plate). The immobilized Pseudo-ICE or ICE-Like polypeptide is then mixed with a Pseudo-ICE or ICE-Like binding molecule under conditions that allows the Pseudo-ICE or ICE-Like binding molecule to interact with the immobilized Pseudo-ICE or ICE-Like polypeptide to form a binding complex. Preferably, the Pseudo-ICE or ICE-Like binding molecule is covalently bound to a detectable moiety, such as an enzyme that converts a colorless substrate into a colored product, which allows monitoring of the interaction between the Pseudo-ICE or ICE-Like polypeptide and its binding molecule. The binding complex is then incubated with a sample that may contain a protein that competitively binds to the Pseudo-ICE or ICE-Like polypeptide. Such competitive binding may be detected by measuring the dissociation of the Pseudo-ICE or ICE-Like binding molecule via its detectable moiety.

Interaction Cloning

Any cloning methods that involve protein-protein interactions can be used for detecting or identifying proteins or polypeptides that specifically interact with, or bind to a Pseudo-ICE or ICE-Like polypeptide. For example, a recombinant Pseudo-ICE or ICE-Like protein of the invention may be purified and radioactively labeled. Preferably, the Pseudo-ICE or ICE-Like protein has a polypeptide tag fused to it to facilitate its purification. A cDNA expression library that may contain a Pseudo-ICE or ICE-Like binding protein is plated and transferred onto nitrocellulose membranes. The membranes are then probed with radioactively labeled, purified recombinant Pseudo-ICE or ICE-Like protein, and washed. Clones that interact with the Pseudo-ICE or ICE-Like protein can be identified by radiography. The cDNA insert encoding a Pseudo-ICE- or ICE-Like-associated protein is then sequenced, and the amino acid sequence of the protein is deduced.

Coimmunoprecipitation

Coimmunoprecipitation assays are well known and can also be used to detect proteins that interact with a Pseudo-ICE or ICE-Like polypeptide or its functional fragment of this invention. Briefly, a Pseudo-ICE or ICE-Like polypeptide or its functional fragment is incubated with a sample that may contain a Pseudo-ICE or ICE-Like binding protein. The mixture may be precleared with normal antiserum and protein A-Sepharose before immuoprecipitation. Immunoprecipitation can be performed by several methods such as by incubating protein A-Sepharose beads pre-adsorbed with anti-Pseudo-ICE or anti-ICE-Like antibody and precipitating the beads. After extensive washing, beads are boiled in Laemmli buffer and the eluted proteins are subjected to SDS-PAGE analysis. The proteins on the SDS-PAGE may be detected by dyes specific to proteins or by labels on the proteins if the proteins in the sample are labeled before incubation with the Pseudo-ICE or ICE-Like polypeptide.

Two-Hybrid System

Alternatively, Pseudo-ICE or ICE-Like binding proteins may be identified by a two-hybrid screening system, such as a yeast two-hybrid system (see Sambrook and Russell, supra). Briefly, in a yeast two-hybrid system, a fusion of nucleic acids encoding a DNA-binding domain of a transcription factor and a Pseudo-ICE or ICE-Like polypeptide or functional fragment thereof (e.g., GAL4 DNA binding domain-Pseudo-ICE fusion or GAL4 DNA binding domain-ICE-Like fusion) is constructed and transformed into a yeast cell containing a GAL binding site linked to a selectable marker gene. A library of cDNAs derived from an organism that may contain Pseudo-ICE or ICE-Like binding proteins and fused to GAL4 activation domain is also constructed and co-transformed. When the cDNA in cDNA-GAL 4 activation domain fusion encodes a polypeptide that interacts with the Pseudo-ICE or ICE-Like or its functional fragment, the selectable marker is expressed. Cells containing the cDNA are then grown, the construct isolated and characterized. The interaction between the polypeptides encoded by the isolated clones and the Pseudo-ICE or ICE-Like polypeptide or Pseudo-ICE or ICE-Like fragment may be verified by in vitro protein-protein interaction assays or communoprecipitation described supra.

Chromotography

Proteins that interact with a Pseudo-ICE or ICE-Like polypeptide or functional fragment thereof of this invention may also be identified or detected by various chromatography techniques. For instance, after incubation of a Pseudo-ICE or ICE-Like polypeptide or functional fragment with a sample that may contain Pseudo-ICE or ICE-Like binding proteins, the mixture may be fractionated on various columns with a salt gradient or the like. In certain embodiments, cell extracts may be directly applied to chromotographic columns if the extracts contain an endogenous Pseudo-ICE or ICE-Like polypeptide. The fractions that contain the Pseudo-ICE or ICE-Like polypeptide or functional fragment thereof can be identified using, e.g., Pseudo-ICE or ICE-Like antibodies, and further characterized by SDS-PAGE analysis. The polypeptides other than the Pseudo-ICE or ICE-Like polypeptide or its fragment in those fractions may be further purified and sequences. The interaction between the identified polypeptides and the Pseudo-ICE or ICE-Like polypeptide or the fragment of Pseudo-ICE or ICE-Like may be verified by any other known protein-protein interaction assays.

The identified protein that binds to Pseudo-ICE or ICE-Like polypeptide or functional fragment thereof may also involve in apoptosis. Such involvement may be tested in any systems where modulation of apoptosis may be assayed (see, e.g., Takayama et al., Cell 80:279-84, 1995; Asai et al., Plant Cell 12:1823-35, 2000). For example, a vector that is able to direct the expression of the nucleic acid encoding the binding protein may be transfected in animal cell lines such as Jurkat T cell lines and 3T3 fibroblasts. Transfected cells with an elevated level of the protein can be detected by its antibodies. Such cells are then subject to several stimuli to induce apoptosis, such as anti-Fas and staurosporine. The survival rate of these cells is compared with that of cells transfected with control vectors that do not direct the expression of the binding protein to detect any apoptotic effect of the protein. In certain embodiments, the above transfected cells may be further transfected with a vector that directs the expression of the Pseudo-ICE or ICE-Like polypeptide or its functional fragment to which the binding protein binds.

The identified protein that specifically binds to a Pseudo-ICE or ICE-Like or its functional fragment may also involve in Pseudo-ICE or ICE-Like mediated processes such as inhibition of pro-caspase-1 activation, inhibition of IL-1β secretion induced by an inflammatory stimulus, and NF-κB activation. Determination of whether the binding protein or another compound or composition (i.e., a candidate effector) is involved in the above processes may be performed by (a) contacting a cell transfected with an expression vector encoding a Pseudo-ICE or ICE-Like polypeptide or a functional fragment of the Pseudo-ICE or ICE-Like polypeptide with the binding protein or the other compound or composition and (b) detecting changes in these processes with the processes in the transfected cell in the absence of the identified protein or the candidate effector as a reference. Detection of changes in Pseudo-ICE or ICE-Like mediated processes may be carried out by any methods known in the art. For example, the effect of the identified protein or the candidate effector on Pseudo-ICE or ICE-Like mediated inhibition of pro-caspase-1 activation may be measured by detecting the level of pro-caspase-1 oligomerization, the processing of pro-caspase-1, enzymatic activities of caspase-1 processed from pro-caspase-1, or IL-1β secretion induced by an inflammatory stimulus such as an IFN-γ, a TNF or a lipopolysaccharide. Exemplary methods are described in detail in Examples and may also be found in Humke et al., Cell 103:99-111 (2000), incorporated herein in its entirety.

Alternatively, determination of whether a candidate effector is involved in Pseudo-ICE or ICE-Like mediated processes may be performed using an in vitro assay. For instance, an effector of Pseudo-ICE or ICE-Like mediated inhibition of pro-caspase-1 activation may be carried out using a method comprising the following steps: (a) forming a mixture of a pro-caspase-1 (or the prodomain of a pro-caspase-1) and Pseudo-ICE (or ICE-Like) or a fragment thereof; (b) contacting the mixture of step (a) with a candidate effector; and (c) detecting changes in the level of pro-caspase-1 activation with that in the absence of the candidate effector as a reference. For the screening for an effector of Pseudo-ICE mediated inhibition of pro-caspase-1 activation, a RICK may also be included in the mixture of step (a).

Certain effectors of Pseudo-ICE or ICE-Like mediated processes may modulate these processes by modulating (enhancing, reducing, or disrupting) the interaction between Pseudo-ICE or ICE-Like polypeptide and another protein that binds to Pseudo-ICE or ICE-Like. In certain preferred embodiments, such effectors are small molecules. In other preferred embodiments, such effectors are polypeptides or fragments thereof. Candidate effectors may be isolated or procured from a variety of sources, such as bacteria, fungi, plants, parasites, libraries of chemicals, random peptides or the like.

In certain embodiments, the present method compares the specific binding between a Pseudo-ICE or ICE-Like polypeptide (or a functional fragment of a Pseudo-ICE or ICE-Like polypeptide) and a Pseudo-ICE or ICE-Like binding protein (e.g., a pro-caspase-1 or a pro-domain of a pro-caspase-1 that interacts with both Pseudo-ICE and ICE-Like polypeptide, and RICK that interacts with Pseudo-Like, but not with ICE-Like) in the absence of a candidate compound with that in the presence of the candidate compound. The increase of the specific binding in the presence of the compound indicates that the compound is able to facilitate the interaction between the Pseudo-ICE or ICE-Like polypeptide and the Pseudo-ICE or ICE-Like binding protein, while the decrease of the specific binding in the presence of the compound indicates that the compound is able to interfere with the interaction between the Pseudo-ICE or ICE-Like polypeptide and the Pseudo-ICE or ICE-Like binding protein.

In some related embodiments, the present method detects the dissociation of either a Pseudo-ICE or ICE-Like polypeptide or a Pseudo-ICE or ICE-Like binding protein from a binding complex between the Pseudo-ICE or ICE-Like polypeptide and the Pseudo-ICE or ICE-Like binding protein in the presence of a candidate compound. Such a dissociation of the Pseudo-ICE or ICE-Like polypeptide or the Pseudo-ICE or ICE-Like binding protein from the binding complex indicates that the compound is capable of disrupting the binding complex between the Pseudo-ICE or ICE-Like polypeptide and the Pseudo-ICE or ICE-Like binding protein.

The characterization of the binding between a Pseudo-ICE or ICE-Like polypeptide and a Pseudo-ICE or ICE-Like binding protein may be performed by any of the techniques known in the art that detect and/or characterize protein-protein interactions. Exemplary techniques are described above in relation to methods of identifying a Pseudo-ICE or ICE-Like binding protein. In certain preferred embodiments, the Pseudo-ICE or ICE-Like polypeptide or its functional fragment is covalently bound to a detectable moiety, such as a reporter molecule and a raionuclide. Also preferred is that the Pseudo-ICE or ICE-Like polypeptide or the Pseudo-ICE or ICE-Like binding protein is attached to a solid support either before or after it forms a binding complex with its binding partner (i.e., the Pseudo-ICE or ICE-Like binding protein for the Pseudo-ICE or ICE-Like polypeptide or the Pseudo-ICE or ICE-Like polypeptide for the Pseudo-ICE or ICE-Like binding protein).

From the foregoing it will be appreciated that, although specific embodiments of the invention have been described herein for purposes of illustration, various modifications may be made without deviating from the spirit and scope of the invention. Accordingly, the invention is not limited except as by the appended claims.

### EXAMPLES

### Example 1

### Isolation and Characterization of Pseudo-Ice and ICD-Like cDNAs

The nucleotide sequences of Pseudo-ICE and ICE-Like cDNAs were identified by searching the GenBank expressed sequence tag (EST) databases for sequences similar to the prodomain of human pro-caspase-1/ICE-α (residues 1-103) using the tBLASTn program. Two EST clones whose accession numbers are AA070591 and AA046000 corresponding to Pseudo-ICE and ICE-like, respectively, were identified and obtained from the I.M.A.G.E consortium (Washington University School of Medicine, St Louis, Mo.) and their entire nucleotide sequence was determined by automated sequencing.

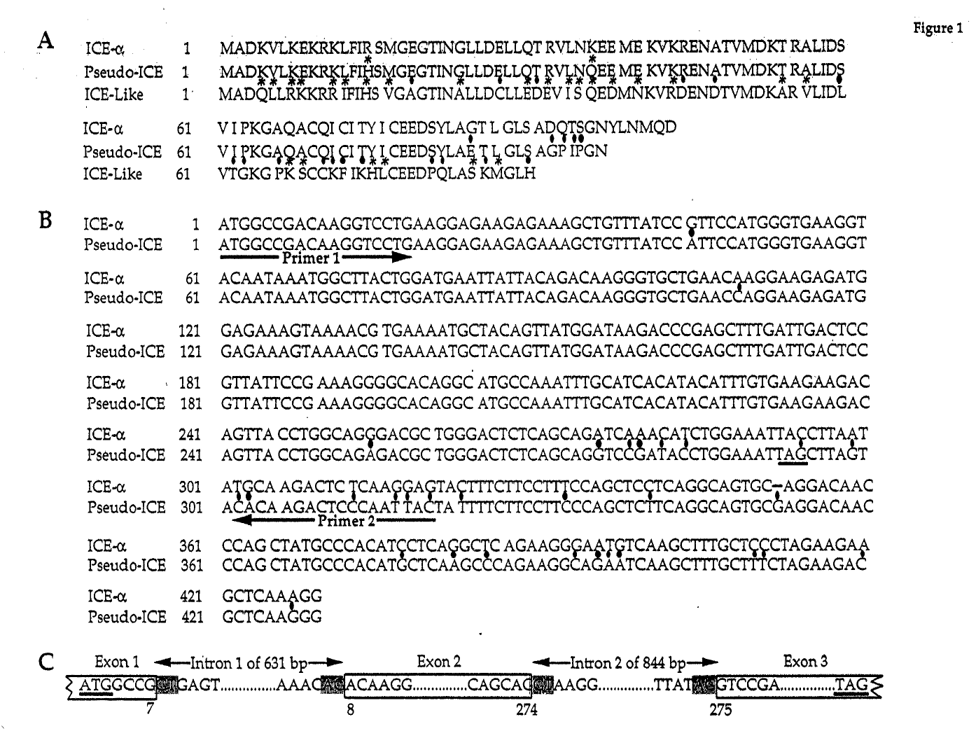

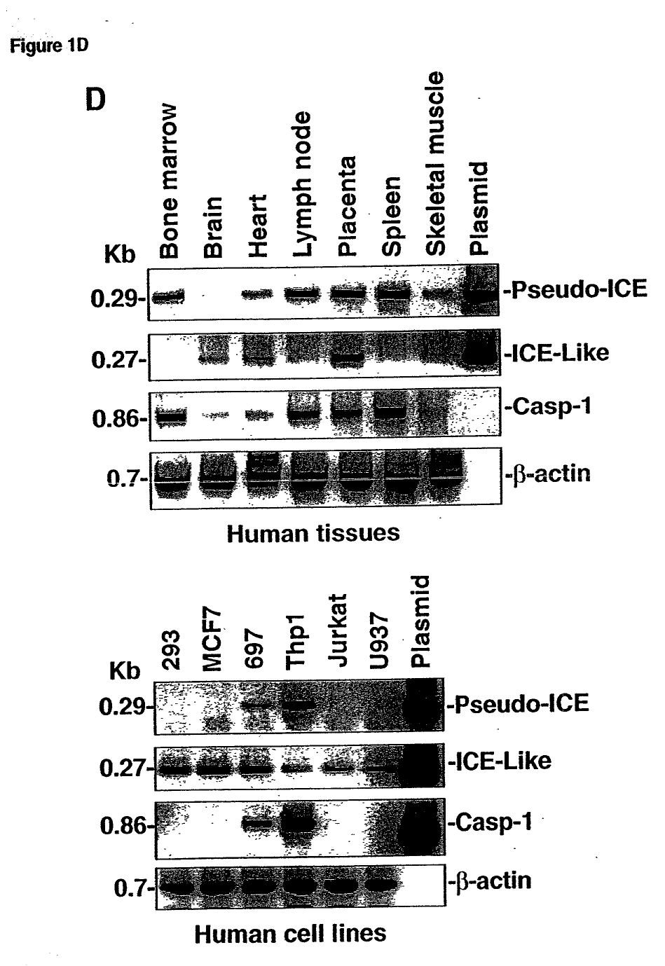

Based on the sequence of the EST clone AA070591, primers unique to Pseudo-ICE were designed (Panel B of FIG. 1) and used in PCR amplification from different human tissues and cell lines. Pseudo-ICE cDNAs were cloned into pcDNA3 (In Vitrogen, Carlsbad, Calif.), and sequenced. Similar procedures were performed to isolate and verify the sequence of ICE-Like cDNA.

ICE-Like cDNA contains a 273 bp open reading frame that codes for a 90-residue protein with a predicted molecular mass of 10.1 kDa and was designated ICE-Like. Multiple sequence alignment of a pro-domain of human pro-caspase-1/ICE-α, Pseudo-ICE, and ICE-Like is shown in Panel A of FIG. 1 where conserved residues and non-conserved substitutions are indicated by stars and dots, respectively. ICE-Like amino acid sequence is 53% identical to the N-terminal sequence of human pro-caspase-1. ICE-Like is identical to ICEBERG (Humke et al., Cell 103:99-111, 2000). Colinear alignment of a pro-domain of human pro-caspase-1/ICE-α and Pseudo-ICE nucleotide sequences, including the 3′ untranslated region of Pseudo-ICE is shown in Panel B of FIG. 1 where stop codon of Pseudo-ICE is underlined and base differences is indicated by dots.

Pseudo-ICE contains a 294 bp open reading frame as evident from the presence of a stop codon upstream of the initiator ATG (data not shown). This sequence encodes a 97-residue protein, named Pseudo-ICE, whose expected molecular weight is 10.7 kDa (Panel A of FIG. 1). Nucleic acid sequences of Pseudo-ICE and pro-caspase-1 prodomain are 97% identical. Pseudo-ICE is about 92% identical to a pro-domain of pro-caspase-1 at the amino acid level (Panel A of FIG. 1).

GenBank High Throughput Genomic Sequences (HTGS) were searched for putative chromosomal localization of Pseudo-ICE and ICE-Like genes. Pseudo-ICE sequence matched with three HTGS clones (GenBank accession numbers AC027011, AP001153, and AP002787) that are mapped to locus 11q22, suggesting that Pseudo-ICE gene is located on chromosome 11. The comparison between Pseudo-ICE sequence and the working draft sequence of HTGS clones revealed that Pseudo-ICE gene is composed of at least 3 exons (Panel C of FIG. 1). In this panel, exons are boxed, 5′ splicing donor and 3′ splicing acceptor are shaded, and the ATG and the stop codon are underlined. ICE-Like sequence also matched two HTGS clones on chromosome 11q22 (AC027011 and AC023068). Genes encoding for caspases 1, 4 and 5 are also found on the same chromosomal locus.

### Example 2

### Expression of Pseudo-ICE AND ICE-Like in Normal and Tumor Cell Lines

The tissue distribution of Pseudo-ICE and ICE-Like in various normal human tissue and tumor cell line mRNA samples was examined by Reverse Transcriptase-Polymerase Chain Reaction (RT-PCR) analysis. RT-PCR was performed to determine the tissue distribution of Pseudo-ICE mRNA using a primer (primer 2) complementary to the 3′ untranslated region of Pseudo-ICE cDNA which differs from pro-caspase-1 sequence and another primer corresponding to the first 18 bp (primer 1) of Pseudo-ICE open reading frame (Panel B of FIG. 1). RT-PCR analysis of multiple tissues and cell line mRNAs with primers 1 and 2 revealed that Pseudo-ICE is expressed mainly in placenta, spleen, lymph node and bone marrow tissues and in the monocytic THP-1 cell line (Panel D of FIG. 1). Sequence analysis of the RT-PCR products from the positive tissues and the THP1 cell line revealed an entire open reading frame identical to that of Pseudo-ICE. Identical distribution was observed for pro-caspase-1, suggesting that pseudo-ICE and pro-caspase-1 are under similar transcriptional regulation. In contrast to pro-caspase-1 and Pseudo-ICE, ICE-Like mRNA was detected mainly in placenta and to a lesser extent in heart (Panel D of FIG. 1), consistently with Humke et al. observations (supra). ICE-Like mRNA was also found in many human cell lines, such as 293, MCF7, 697, THP-1, Jurkat, and U937 cell lines (Panel D of FIG. 1).

### Example 3

### Homo- AND Hetero-Dimerization of Pseudo-ICE and ICE-Like

Whether Pseudo-ICE and ICE-Like can bind to pro-caspase-1 or to RICK was determined using both in vitro and in vivo assays as described below.

Origin and Construction of Expression and Reporter Plasmids

The entire reading frame of Pseudo-ICE, ICE-Like and pro-caspase-1 (Alnemri et al., J. Biol. Chem. 270:4312, 1995) were amplified by PCR using complementary PCR adaptor primers spanning the initiation and stop codons of these genes.

T7, Flag, enhanced green-fluorescent protein (EGFP) and gluthatione-S-transferase (GST)-fused genes were made by cloning PCR products in-frame into the expression vector pcDNA3 (In Vitrogen), pCMV2 (Sigma, Saint-Louis, Mo.), pEGFP-C1, pEGFP-N1 (Clontech, Palo Alto, Calif.) and pGEX-5X 3 (Amersham Pharmacia Biotech, San Francisco, Calif.), respectively. T7-tagged cDNAs for Pseudo-ICE, ICE-Like and pro-caspase-1 C to A active site mutant, were generated by PCR and subcloned into pMSCVneo retroviral vector (Clontech).

Expression vectors for human Apaf-1, cCLAP, CRADD, pro-caspase-8, pro-caspase-10, and pro-caspase-9 C 287 A have been described before (Li et al., Cell 91:479 (1997); MacFarlane et al., J. Biol. Chem. 272:25417 (1997); Ahmad et al., Cancer Res. 57:615 (1997); Srinivasula et al., J. Biol. Chem. 274:17946 (1999)). RICK expression contruct was a generous gift from Dr. G. Nunez (University of Michigan, Ann Harbor, Mich.). Kinase inactive mutant of IκB kinase (IKK)-α were kindly provided by Dr. W. C. Greene (University of California, San Francisco, Calif.). The 5×kB-luciferase reporter plasmid was purchased from Stratagene (La Jolla, Calif.). All other cDNAs used in this study were generated by PCR using appropriate PCR primers and subcloned into appropriate expression constructs.

GST Fusion Proteins and In Vitro Interaction Assay

The GST fusion proteins were expressed in E. coli DH5-α bacteria (Life Technologies, Rockville, Md.) incubated for 2 h at 30° C. in the presence of 0.5 mM of isopropyl-1-thio-β-D-galactoside (IPTG). Cells were sonicated in GST buffer composed of 20 mM N-(2-hydroxyethyl)pauperizing-N′-(2-ethanesulfonic acid) (HEPES), pH 7.6, 1.5 mM MgCl₂, 100 mM NaCl, 1 mM dithiotreitol (DTT), 1% Triton-X100 and phenyl-methyl-sulfonyl-fluoride (PMSF). Cells were then centrifuged at 10000 g for 15 min and supernatants were incubated for 2 h at 4° C. with gluthatione Sepharose 4B resin (Amersham Pharmacia Biotech). The protein-bound resin (30 μl) was washed with GST buffer and incubated overnight at 4° C. in the presence of 5 μl of ³⁵S-labeled in vitro-translated products. Labeled proteins were prepared from pcDNA3 constructs by in vitro transcription and translation in the presence of ³⁵S-methionine using T7-coupled transcription/translation TNT kit according to the manufacturer's instructions (Promega, Madison, Wis.). Samples of the protein-bound resins were washed three times in GST buffer, boiled in sodium dodecyl sulfate (SDS) Laemmli sample buffer and fractionated by electrophoresis on an SDS-polyacrylamide gel. Proteins were then visualized by autoradiography.

In Vivo Interaction Assay

Human embryonic kidney 293 T cells, were cultured in 100 nm dishes in Dulbecco modified essential medium (DMEM) supplemented with 10% fetal bovine serum (FBS) and antibiotics (Life Technologies). Cells at 50% confluency, were transiently co-transfected with equal amounts of T7-pcDNA3 and Flag-pCMV2 constructs (total DNA of 10 μg/dish) in OptiMEM using the lipofectamine method according to the manufacturer's recommendations (Life Technologies). Twelve hours after transfection, OptiMEM was replaced with DMEM/10% FBS. Twenty four hours later, cells were lysed in 300 μl of a buffer containing 25 mM tris(hydroxymethylaminomethane), pH 7.6, 150 mM NaCl, 1% nonidet-P40, and PMSF. Samples were centrifuged. One third of the supernatant was put aside to verify by Western blot the expression of the proteins. The remaining 200 μl of supernatant were incubated for 2 h at 4° C. with anti-Flag M5 plus M2 monoclonal antibodies (Ab; Sigma). The immune complexes were precipitated overnight at 4° C. with protein-G Sepharose (Amersham-Pharmacia Biotech), washed extensively and then eluted by boiling in SDS-sample buffer. The eluted proteins were resolved by SDS-PAGE and detected by Western blot analysis with horseradish peroxidase (HRP)-conjugated anti-T7 Ab (Novagen, Madison, Wis.). Bands were then detected using enhanced chemoluminescence kit (Amersham Pharmacia Biotech).

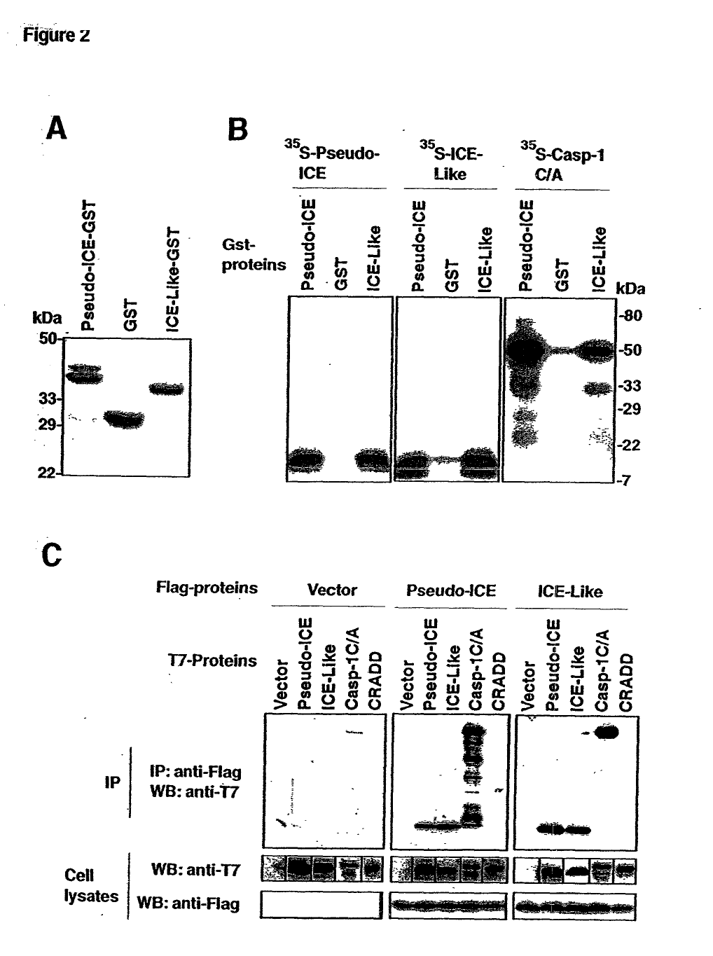

The results of in vitro and in vivo interactions of Pseudo-ICE and ICE-Like with pro-caspase-1 are shown in FIG. 2. In the in vitro assay, ³⁵S-labeled pro-caspase-1 was incubated with GST, GST-conjugated Pseudo-ICE or ICE-Like bound to resin (FIG. 2A). GST-conjugated Pseudo-ICE or ICE-Like, but not GST alone, were able to interact strongly with ³⁵S-labeled pro-caspase-1 in vitro (Panel B of FIG. 2). GST-Pseudo-ICE and ICE-Like were also able to interact with ³⁵S-labeled Pseudo-ICE and ICE-Like, suggesting that these proteins can self associate and can interact with each other (Panel B of FIG. 2). In vivo binding assays also confirmed the ability of Pseudo-ICE and ICE-Like to self-associate (Panel C of FIG. 2).

The interactions described above are specific since neither Pseudo-ICE, nor ICE-Like co-precipitated in vitro or in vivo other CARD-containing proteins tested, comprising pro-caspase-2,-4, and -9, Apaf-1, CRADD, c-CLAP, and c-IAP2 (Panel C of FIG. 2 and data not shown). It has been found that Pseudo-ICE and ICE-Like do not interact either with pro-caspase-8, or -10, or with FADD, which contain DEDs and/or death domains structurally and functionally related to CARD (data not shown, Hofmann, Cell. Mol. Life Sci. 55:1113, 1999).

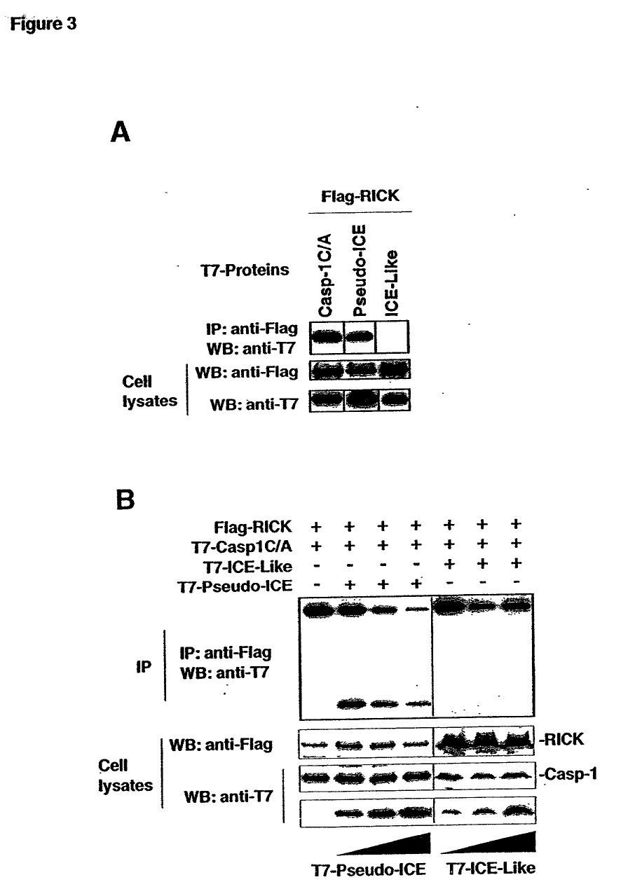

The results of in vitro and in vivo assays of Pseudo-ICE and ICE-Like with RICK are shown in FIG. 3. For the in vitro assay, 293 T cells were transfected with Flag-tagged RICK and T7-tagged Pseudo-ICE or ICE-Like and immunoprecipitation was performed with an anti-Flag Ab. As shown in Panel A of FIG. 3, pro-caspase-1 and Pseudo-ICE but not ICE-Like were able to interact with RICK. Similar results were obtained by in vitro interaction assays with ³⁵S-labeled RICK and GST fusion proteins of caspase-1-CARD, Pseudo-ICE or ICE-Like (data not shown). The ability of Pseudo-ICE to interact with RICK was expected based on the high degree of homology between the CARD of pro-caspase-1, which mediates its interaction with RICK, and Pseudo-ICE. The absence of any interaction between ICE-Like and RICK is consistent with the results described recently by Humke et al. (supra).

To determine whether expression of Pseudo-ICE and ICE-Like affects the interaction between RICK and pro-caspase-1, in vivo interaction assays between RICK and pro-caspase-1 in the presence or absence of increasing amounts of Pseudo-ICE and ICE-Like were performed. 273 T cells were cotransfected with constructs encoding Flag-RICK (2.5 μg) and T7-caspase 1 C to A (2 μg) together with increasing amounts (2, 5, 8 μg) of T7 Pseudo-ICE or T7-ICE-Like constructs. Both Pseudo-ICE and ICE-Like prevented binding of pro-caspase-1 to RICK in a dose dependent manner, although the effect of Pseudo-ICE was more dramatic (Panel B of FIG. 3). These results indicate that the CARD-CARD interactions between pro-caspase-1 and Pseudo-ICE or ICE-like interfere with binding of pro-caspase-1 to RICK. Moreover, the ability of Pseudo-ICE to interact with RICK could further decrease the binding of pro-caspase-1 to RICK.

### Example 4

### Effects of Pseudo-ICE and ICE-Like on Induced Secretion of IL-1β

The effects of Pseudo-ICE and ICE-Like on the induced secretion of IL-1β in THP-1 monocytic cell line was investigated as described below. The monocytic cell line THP-1 was cultured in RPMI 1640 supplemented with 10 mM HEPES, 1 mM sodium pyruvate, 55 μM β-mercaptoethanol, 10% FBS, antibiotics (all from Life Technologies) and 5 ng/ml macrophage-colony stimulating factor (Sigma). The amphotropic packaging cell line Phoenix (G. P. Nolan's laboratory, Stanford University Medical Center, Stanford, Calif.) was transfected with pMSCV neo constructs using the calcium phosphate/chloroquine method (Kinsella and Nolan, Hum. Gene Ther. 7:1405, 1996). Forty eight hours after transfection, culture supernatants containing retroviral particles were collected and filtered through 0.45 μm membrane. THP-1 cells (1×10⁶ cells/well) were then centrifuged in 6 well plates for 45 min at 1800 rpm at 32° C. in the presence of 3 ml of retrovirus enriched supernatant supplemented in 4 μg/ml polybrene (Sigma). Plates were placed back in a CO₂ incubator at 32° C. for 1.5 h. Supernatants were replaced for fresh retrovirus enriched medium. THP-1 cells were subjected to a total of three cycles of infection followed by 48 h of culture in THP-1 growth medium described above. Cells were then selected using 1 mg/ml neomycin (Life Technologies). After two weeks of culture, viable THP-1 cells were sedimented and cultured in 96 well plates in the presence of neomycin.

After several weeks of culture, the expression of transgenes was verified by Western blotting of mass culture lysates.

To assay for IL-1β and IL-1ra, cells (0.75×10⁶ cells/ml) were cultured for 24 h in THP-1 medium without FBS and macrophage colony-stimulating factor and were incubated for 4 h with 100 units/ml of IFN-γ and then for 18 h with 10 μg/ml of LPS. Supernatants diluted at 1/2-1/4 or 1/10-1/40 were used to quantify IL-1β or IL-1ra, respectively, by enzyme-linked immunosorbent assay (R&D systems, Minneapolis, Minn.).

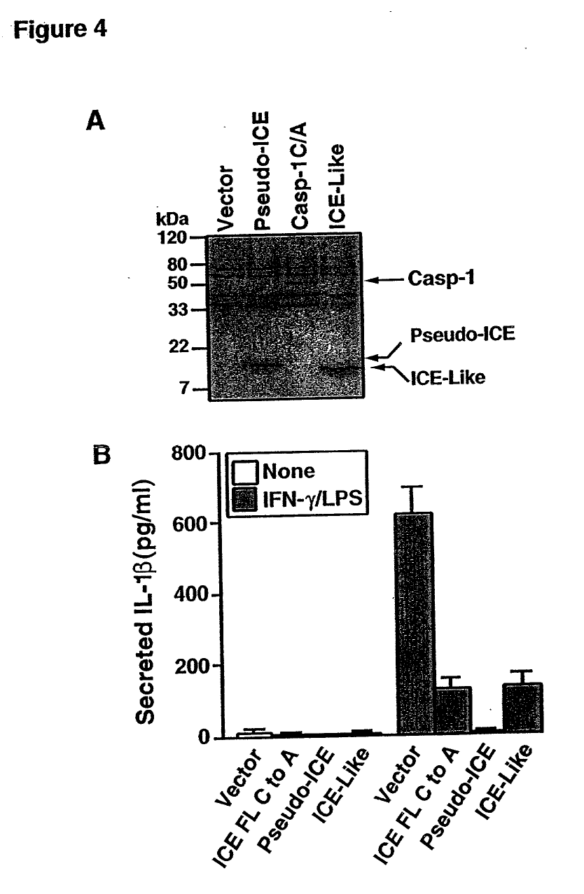

As shown in Panel A of FIG. 4, all stable transfectants expressed detectable amounts of Pseudo-ICE, ICE-like and pro-caspase-1 C to A. Interestingly, expression of pro-caspase-1 C/A or ICE-Like reduce by around 80% the secretion of IL-1β, whereas expression of Pseudo-ICE completely blocked the release of IL-1β in response to IFN-γ and LPS, suggesting strongly that these proteins block specifically ICE activation (Panel B of FIG. 4). No significant effect by these proteins was observed on secretion of IL-1ra (data not shown), another cytokine that is secreted from THP-1 in response to IFN-γ and LPS (Kline et al., Am. J. Physiol. 269:L92, 1995). Furthermore, treatment with IFN-γ and LPS of the same number of uninfected or mock-infected (vector) THP-1 cells induces the secretion of the same amount of IL-1β, i.e. 585.2±102.7 and 629.8±69.1 pg/ml, respectively (mean ±SEM, n=7 and 3, respectively). These results indicate that retroviral infection followed by selection has not modified the ability of THP-1 cells to respond to IFN-γ and LPS. Taken together, the results demonstrate that Pseudo-ICE and ICE-Like reduce IL-1β secretion, probably by binding to the prodomain of pro-caspase-1 thereby preventing its activation by RICK or another novel pro-caspase-1 activator.

### Example 5

### Apoptotic Activity of Pseudo-ICE and ICE-Like

Many regulators of apoptosis such as the death receptors and the adaptor molecules, FADD, CRADD, RIP, induce apoptosis when overexpressed in the absence of additional apoptotic signals. In addition, the ectopic expression of the prodomain of pro-caspase-1 was shown to induce cell death in MCF-7 cells presumably because of the ability of the pro-caspase-1 N-terminal end to translocate to the nucleus (Mao et al., J. Biol. Chem. 273:16968, 1998). To examine the apoptotic activity of Pseudo-ICE and ICE-Like, MCF-7 Fas cells, breast adenocarcinoma cells stably transfected with Fas receptor, were seeded in triplicate at 0.05×10⁶ cells/well in six well plates in DMEM/10% FBS. Thirty six hours later, cells were transfected with 0.2 μg pRSC LacZ reporter plasmid, 0.8 μg T7 Pseudo-ICE, ICE-Like, or pro-caspase-9 FL C 287 A together with 4 μl of lipofectamine in 1 ml of OptiMEM. Eight hours after the beginning of transfection, OptiMEM medium was replaced with DMEM/10% FBS with and without 2 or 10 ng/ml of tumor necrosis factor (TNF)-α added to the culture, for 12 h. Cells were then stained in situ for β-galactosidase activity using the β-Gal staining kit from Invitrogen. The proportions of apoptotic cells were evaluated by counting the numbers of stained normal and apoptotic cells. The cells from the third remaining well were collected and lysed. Cell lysates were used to verify the expression of the transgenes.

In addition, intracellular localization of Pseudo-ICE or ICE-Like was also determined using the method described below. MCF-7 Fas cells were transfected with 1 μg of Pseudo-ICE or ICE-Like pEGFP-C1 or -N1 as described in Example 3. Twenty-four hours after transfection, the intracellular localization was evaluated by observing the cells using a fluorescence microscope.

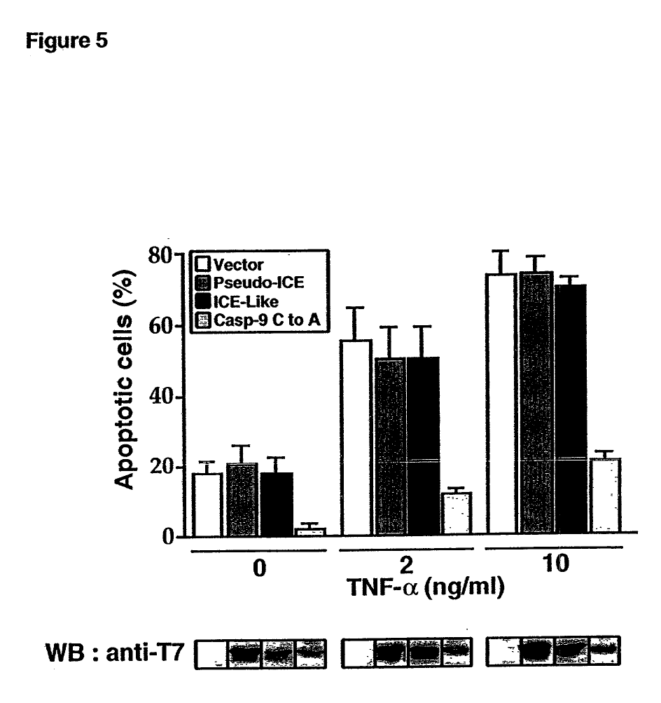

The results of the above apoptosis assay indicate that Pseudo-ICE does not induce apoptosis when overexpressed in MCF-7 Fas cells and does not affect TNF-α-induced apoptosis (FIG. 5). Similarly to Pseudo-ICE and unlike to caspase-9 C to A, ICE-Like ectopic expression has no effects on basal or TNF-α-induced cell death in MCF-7 Fas cells (FIG. 5). Moreover, transiently expressed Pseudo-ICE and ICE-Like conjugated in N or C terminal of EGFP are evenly distributed in the cytoplasm, but not in the nucleus, of MCF-7 Fas cells treated or not by TNF-α (data not shown). Interestingly, in a few cells, cytoplasmic filaments were observed (data not shown). Similar filaments have been described in cells overexpressing members of the superfamily of six helix bundle interaction domain such as caspase-8 DED, FADD DED and c-CLAP CARD (Hofmann, Cell. Mol. Life Sci. 55:1113, 1999; Guiet and Vito, J. Cell Biol. 148:1131; Siegel et al., J. Cell Biol. 141:1243, 1998).

### Example 6

### NF-κB Regulating Activity of Pseudo-ICE and ICE-Like

The possibility that Pseudo-ICE and ICE-Like may activate NF-κB was examined by the method described below. 293 T cells were seeded in triplicate at 0.3×10⁶ cells/well in twelve well plates in DMEM/10% FBS. Twenty-four hours later, cells were transfected with various expression constructs encoding T7- or Flag-epitope tagged proteins plus 0.025 μg 5×κB-luciferase and 0.2 μg pRSC-LacZ reporter plasmids, together with 3 μl of lipofectamine in 1 ml of OptiMEM. In each well the total amount of DNA was kept constant at 0.7 μg by addition of empty vector pcDNA3 or pCMV2. OptiMEM medium was replaced for DMEM/10% FBS 8 h after transfection. Sixteen hours after the change of medium, the cells were collected, washed in phosphate buffer saline and lysed using lysis buffer from Promega's luciferase assay system kit. In some experiments, cells were treated for 6 h with 20 ng/ml of TNF-α prior to harvesting.

NF-κB activity was assayed on 5 μl of cell lysates by chemiluminescence as per the instructions of Promega. To normalize for transfection efficiency, cell lysates were also subjected to β-galactosidase spectrophotometric assay. In brief, 10 μl of cell lysate were incubated for 15 min at 37° C. with 290 μl of a solution containing 0.9 mg/ml o-nitrophenyl-β-D-galactopyranoside (ONPG), 1 mM MgCl₂, 45 mM β-mercaptoethanol, 100 mM sodium phosphate, pH 7.5. The reaction was stopped by addition of 500 μl of 1M Na₂CO₃. The optical density was read at a wavelength of 420 nm. Results are expressed as relative luciferase units/optical density for β-galactosidase activity.

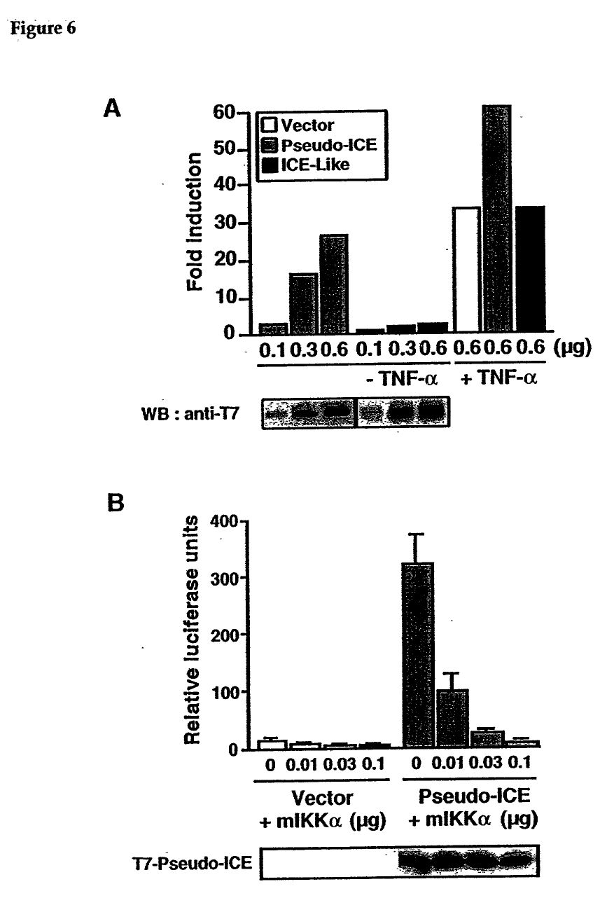

The results of the above assay indicate that Pseudo-ICE strongly activates NF-κB in a dose-dependent manner and enhances TNF-α-induced NF-κB activation (Panel A of FIG. 6). At levels of expression comparable to that of Pseudo-ICE, ICE-Like has very little effect on the basal or TNF-α-induced NF-κB activity (Panel A of FIG. 6). This suggests that NF-κB activity is unlikely to be provoked by a non-specific cellular stress mediated by transient expression of Pseudo-ICE. This stress termed the endoplasmic reticulum-overload response has been described by Pahl et al. (Pahl, Oncogene 18:6853, 1999). Finally, a kinase inactive mutant of IKK-α dose-dependently inhibits NF-κB activity provoked by Pseudo-ICE without modifying its expression as shown in Panel B of FIG. 6. Combined, these results indicate that Pseudo-ICE activates NF-κB via a mechanism dependent on the IKK complex (Karin, Oncogene 18:6867, 1999). Since Pseudo-ICE, but not ICE-Like, interacts with the CARD-containing kinase RICK, it is possible that Pseudo-ICE induces oligomerization of RICK leading to activation of NF-κB. Oligomerization of RIP and RICK, which interact directly with IKK-γ, an essential component of the IKK complex, has been recently proposed as a mechanism by which upstream regulators transmit their activation signals to the IKK complex leading to its activation (Poyet et al., J. Biol. Chem. 275:37966, 2000; Inohara et al., J. Biol. Chem. 275:27823, 2000).

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An isolated nucleic acid molecule comprising a nucleic acid sequence encoding a Pseudo-ICE having at least 80% amino acid identity to SEQ ID NO: 1, an immunogenic fragment of the Pseudo-ICE, or a functional fragment of the Pseudo-ICE that has an activity selected from: specific binding to pro-caspase-1, specific binding to RICK, inhibiting induced IL-1β secretion, and stimulating NF-κB activation.

2. The isolated nucleic acid molecule of claim 1 wherein the Pseudo-ICE has an amino acid sequence set forth in SEQ ID NO: 1.

3. The isolated nucleic acid molecule of claim 1 wherein the nucleic acid sequence has a nucleotide sequence at least 98% identical to SEQ ID NO: 2.

4. The isolated nucleic acid molecule of claim 1 wherein the nucleic acid sequence has a nucleotide sequence as set forth in SEQ ID NO: 2.

5. A nucleic acid vector comprising a nucleic acid sequence encoding a Pseudo-ICE having at least 80% amino acid identity to SEQ ID NO: 1 or a functional fragment of the Pseudo-ICE.

6. The nucleic acid vector of claim 5 wherein the Pseudo-ICE has an amino acid sequence set forth in SEQ ID NO: 1.

7. The nucleic acid vector of claim 5 wherein the nucleic acid sequence has a nucleotide sequence at least 98% identical to SEQ ID NO: 2.

8. The nucleic acid vector of claim 5 wherein the nucleic acid sequence has a nucleotide sequence as set forth in SEQ ID NO: 2.

9. A host cell containing the nucleic acid vector of claim 5.

10. The host cell of claim 9 wherein the host cell is selected from the group consisting of a mammalian cell, a plant cell, an insect cell, a yeast cell and a bacterial cell.

11. An isolated polypeptide comprising a Pseudo-ICE having at least 80% amino acid identity to SEQ ID NO: 1, an immunogenic fragment of the Pseudo-ICE, or a functional fragment of the Pseudo-ICE that has an activity selected from: specific binding to pro-caspase-1, specific binding to RICK, inhibiting induced IL-1β secretion, and stimulating NF-κB activation.

12. The isolated polypeptide of claim 11 wherein the Pseudo-ICE has an amino acid sequence set forth in SEQ ID NO: 1.

13. An isolated polypeptide encoded by a nucleotide sequence at least 98% identical to SEQ ID NO: 2 and having an activity selected from: specific binding to pro-caspase-1, specific binding to RICK, inhibiting induced IL-1β secretion, and stimulating NF-κB activation.

14. An antibody comprising an immunoglobulin or antigen-binding fragment thereof that specifically binds the isolated polypeptide of claim 11 or claim 12.

15. A composition comprising the isolated polypeptide of claim 11 or claim 12 and a physiologically acceptable carrier.

16. A method of transforming or transfecting a cell with a nucleic acid molecule encoding a Pseudo-ICE that has at least 80% amino acid identity to SEQ ID NO: 1 or a functional fragment of the Pseudo-ICE that has an activity selected from: specific binding to pro-caspase-1, specific binding to RICK, inhibiting induced IL-1β secretion, and stimulating NF-κB activation, comprising contacting the cell with a vector comprising the nucleic acid molecule under the control of a promoter.

17. The method of claim 16 wherein the Pseudo-ICE has an amino acid sequence set forth in SEQ ID NO: 1.

18. The method of claim 16 wherein the nucleic acid molecule is at least 98% identical to SEQ ID NO: 2.

19. The method of claim 16 wherein the nucleic acid molecule has a nucleotide sequence set forth in SEQ ID NO: 2.

20. The method of claim 16 wherein the nucleic acid molecule is in the sense orientation with respect to the promoter.

21. The method of claim 16 wherein the nucleic acid molecule is in the antisense orientation with respect to the promoter.

22. A method of inhibiting apoptosis or inflammation comprising contacting a cell expressing a pro-caspase-1 with a composition comprising the isolated polypeptide of claim 11 or claim 12 under conditions and for a time sufficient to permit the inhibition of apoptosis.

23. A method of inhibiting apoptosis or inflammation comprising contacting a cell expressing a pro-caspase-1 with a composition comprising the isolated nucleic acid molecule of any one of claims 1-3 under conditions and for a time sufficient to permit the inhibition of apoptosis.

24. A method of stimulating apoptosis comprising contacting a cell expressing a pro-caspase-1 a composition comprising a polypeptide that specifically binds to the isolated polypeptide of claim 11 or claim 12 under conditions and for a time sufficient to permit the stimulation of apoptosis.

25. The method of claim 24 wherein the polypeptide is an immunoglobulin or antigen-binding fragment thereof that specifically binds to the isolated polypeptide of claim 11 or claim 12.

26. A method of stimulating apoptosis comprising contacting a cell expressing a pro-caspase-1 a composition comprising an antisense or ribozyme construct of the isolated nucleic acid molecule of any one of claims 1-3 under conditions and for a time sufficient to permit the stimulation of apoptosis.

27. A method of inhibiting the activation of a pro-caspase-1 comprising contacting a cell expressing the pro-caspase-1 with a composition comprising the isolated polypeptide of claim 11 or claim 12 under conditions and for a time sufficient to permit the inhibition of the activation of the pro-caspase-1.

28. A method of inhibiting the activation of a pro-caspase-1 comprising contacting a cell expressing the pro-caspase-1 with a composition comprising the isolated nucleic acid molecule of any one of claims 1-3 under conditions and for a time sufficient to permit the inhibition of the activation of the pro-caspase-1.

29. A method of stimulating the activation of a pro-caspase-1 comprising contacting a cell expressing the pro-caspase-1 a composition comprising a polypeptide that specifically binds to the isolated polypeptide of claim 11 or claim 12 under conditions and for a time sufficient to permit the stimulation of the activation of the pro-caspase-1.

30. The method of claim 29 wherein the polypeptide is an immunoglobulin or antigen-binding fragment thereof that specifically binds to the isolated polypeptide of claim 11 or claim 12.

31. A method of stimulating the activation of a pro-caspase-1 comprising contacting a cell expressing the pro-caspase-1 a composition comprising an antisense or ribozyme construct of the isolated nucleic acid molecule of any one of claims 1-3 under conditions and for a time sufficient to permit the stimulation of the activation of the pro-caspase-1.

32. A method of identifying inhibitors or enhancers of Pseudo-ICE mediated inhibition of pro-caspase-1 activation, comprising:
a. contacting a cell transfected with an expression vector encoding Pseudo-ICE having at least 80% amino acid identity to SEQ ID NO: 1 or a functional fragment of the Pseudo-ICE capable of inhibiting pro-caspase-1 activation with a candidate inhibitor or enhancer; and
b. detecting an increase or decrease in pro-caspase-1 activation in the presence of the candidate inhibitor or enhancer, wherein a decrease in pro-caspase-1 activation indicates the presence of an enhancer of Pseudo-ICE mediated inhibition of pro-caspase-1 activation and an increase in pro-caspase-1 activation indicates the presence of an enhancer.

33. The method of claim 32 wherein step (b) detects the level of pro-caspase-1 oligomerization.

34. The method of claim 32 wherein step (b) detects the level of the pro-caspase-1 processing activity.

35. The method of claim 32 wherein step (b) detects the substrate cleavage activity of the caspase-1 processed from the pro-caspase-1.

36. The method of claim 32 wherein step (b) detects the level of IL-1β secretion induced by an inflammatory stimulus.

37. A method of inhibiting induced secretion of IL-1β comprising contacting a cell subject to an inflammatory stimulus that induces IL-1β secretion with a composition comprising the isolated polypeptide of claim 11 or claim 12 under conditions and for a time sufficient to permit the inhibition of IL-1β secretion induced by the stimulus.

38. A method of inhibiting induced secretion of IL-1β comprising contacting a cell subject to an inflammatory stimulus that induces IL-1β secretion with a composition comprising the isolated nucleic acid molecule of any one of claims 1-3 under conditions and for a time sufficient to permit the inhibition of IL-1β secretion induced by the stimulus.

39. The method of claim 37 or claim 38 wherein the inflammatory stimulus is an IFN-γ, a TNF, or a lipopolysaccharide.

40. A method of identifying inhibitors or enhancers of Pseudo-ICE mediated inhibition of IL-1β secretion in response to an inflammatory stimulus, comprising:
a. contacting a cell transfected with an expression vector encoding Pseudo-ICE having at least 80% amino acid identity to SEQ ID NO: 1 or a functional fragment of the Pseudo-ICE capable of inhibiting the IL-1β secretion with a candidate inhibitor or enhancer;
b. subjecting the transfected cell to an inflammatory stimulus; and
c. detecting an increase or decrease in the IL-1β secretion level induced by the inflammatory stimulus in the presence of the candidate inhibitor or enhancer, wherein a decrease in the induced IL-1β secretion indicates the presence of an enhancer of Pseudo-ICE mediated inhibition of the induced IL-1β secretion and an increase in the induced IL-1β secretion indicates the presence of an inhibitor of Pseudo-ICE mediated inhibition of the induced IL-1β secretion.

41. The method of claim 40 wherein the inflammatory stimulus is an IFN-γ, a TNF, or a lipopolysaccharide.

42. The method of claim 30 wherein the Pseudo-ICE has an amino acid sequence set forth in SEQ ID NO: 1.

43. A method of stimulating the activation of an NF-kB comprising contacting a cell expressing the NF-kB with a composition comprising the isolated polypeptide of claim 11 or claim 12 under conditions and for a time sufficient to permit the inhibition of the activation of the NF-kB.

44. A method of stimulating the activation of an NF-kB comprising contacting a cell expressing the NF-kB with a composition comprising the isolated nucleic acid molecule of any one of claims 1-3 under conditions and for a time sufficient to permit the inhibition of the activation of the NF-kB.

45. A method of inhibiting the activation of an NF-kB comprising contacting a cell expressing the NF-kB a composition comprising a polypeptide that specifically binds to the isolated polypeptide of claim 11 or claim 12 under conditions and for a time sufficient to permit the stimulation of the activation of the NF-kB.

46. The method of claim 45 wherein the polypeptide is an immunoglobulin or antigen-binding fragment thereof that specifically binds to the isolated polypeptide of claim 11 or claim 10.

47. A method of inhibiting the activation of an NF-kB comprising contacting a cell expressing the NF-kB a composition comprising an antisense or ribozyme construct of the isolated nucleic acid molecule of any one of claims 1-3 under conditions and for a time sufficient to permit the stimulation of the activation of the NF-kB.

48. A method of identifying inhibitors or enhancers of Pseudo-ICE mediated NF-kB activation, comprising:
a. contacting a cell transfected with an expression vector encoding Pseudo-ICE having at least 80% amino acid identity to SEQ ID NO: 1 or a functional fragment of the Pseudo-ICE capable of stimulating NF-κB activation with a candidate inhibitor or enhancer; and
b. detecting an increase or decrease in NF-kB activation in the presence of the candidate inhibitor or enhancer, wherein a decrease in NF-kB activation indicates the presence of an inhibitor and an increase in NF-kB indicates the presence of an enhancer.

49. The method of claim 48 wherein the Pseudo-ICE has an amino acid sequence set forth in SEQ ID NO: 1.

50. A method of identifying a polypeptide that specifically binds to a Pseudo-ICE having at least 80% amino acid identity to SEQ ID NO: 1 or a functional fragment of the Pseudo-ICE that has an activity selected from: specific binding to pro-caspase-1, specific binding to RICK, inhibiting induced IL-1β secretion, and stimulating NF-κB activation, comprising:
a. contacting a sample with the Pseudo-ICE or the functional fragment under conditions that permit the formation of a complex between the Pseudo-ICE or the functional fragment thereof and the polypeptide; and
b. detecting the complex and polypeptide in the complex.

51. The method of claim 50 wherein the Pseudo-ICE has an amino acid sequence set forth in SEQ ID NO: 1.

52. The method of claim 50 wherein the Pseudo-ICE or the functional fragment thereof is covalently bound to a detectable moiety.

53. The method of claim 52 wherein the detectable moiety is a reporter molecule.

54. The method of claim 52 wherein the detectable moiety is a radionuclide.

55. The method of claim 50 wherein the sample comprises a cDNA expression library.

56. A nucleic acid molecule comprising a first nucleic acid sequence encoding a Pseudo-ICE having at least 80% identity to SEQ ID NO: 1 or a finctional fragment of the Pseudo-ICE that has an activity selected from: specific binding to pro-caspase-1, specific binding to RICK, inhibiting induced IL-1β secretion, and stimulating NF-κB activation and a second nucleic acid sequence encoding the transcription activation domain or the DNA-binding domain of a transcription factor.

57. A method for identifying a polypeptide that specifically binds to a Pseudo-ICE having at least 80% amino acid identity to SEQ ID NO: 1 or a functional fragment of the Pseudo-ICE that has an activity selected from: specific binding to pro-caspase-1, specific binding to RICK, inhibiting induced IL-1β secretion, and stimulating NF-κB activation with a yeast two-hybrid screening system, comprising transforming a yeast cell with a vector comprising the nucleic acid molecule of claim 56.

58. A method for identifying a compound that increases the specific binding between a pro-caspase-1 or a fragment thereof that comprises a pro-domain of the pro-caspase-1 and a Pseudo-ICE having at least 80% amino acid identity to SEQ ID NO: 1 or a finctional fragment of Pseudo-ICE capable of binding to the pro-caspase-1 or the fragment thereof, comprising:
a. combining the Pseudo-ICE or the functional fragment with the pro-caspase-1 or the fragment thereof in the absence of a candidate compound under conditions that allow specific binding between the Pseudo-ICE or the functional fragment and the pro-capase-1 or the fragment thereof;
b. combining the Pseudo-ICE or the functional fragment with the pro-caspase-1 or the fragment thereof in the presence of the candidate compound under conditions of step (a);
c. comparing the specific binding between the Pseudo-ICE or the functional fragment and the pro-caspase-1 or the fragment thereof of step (a) with that of step (b) to thereby determine whether the candidate compound is capable of increasing the specific binding between the Pseudo-ICE or the functional fragment and the pro-caspase-1 or the fragment thereof.

59. The method of claim 58 wherein the Pseudo-ICE has an amino acid sequence of SEQ ID NO: 1.

60. A process for manufacturing a compound that increases the specific binding between a pro-caspase-1 or a fragment thereof that comprises a pro-domain of the pro-caspase-1 and a Pseudo-ICE having at least 80% amino acid identity to SEQ ID NO: 1 or a functional fragment of Pseudo-ICE capable of binding to the pro-caspase-1 or the fragment thereof, comprising:
a. carrying out the method of claim 58 to identify a compound that increases the specific binding between a pro-caspase-1 or a fragment thereof and a Pseudo-ICE having at least 80% amino acid identity to SEQ ID NO: 1 or a functional fragment of Pseudo-ICE capable of binding to the pro-caspase-1;
b. derivatizing the compound; and
c. optionally repeating steps a and b.

61. The process of claim 60 wherein the Pseudo-ICE has an amino acid sequence of SEQ ID NO: 1.

62. A method for identifying a compound that decreases the specific binding between a pro-caspase-1 or a fragment thereof that comprises a pro-domain of the pro-caspase-1 and a Pseudo-ICE having at least 80% amino acid identity to SEQ ID NO: 1 or a functional fragment of Pseudo-ICE capable of binding to a pro-caspase-1, comprising:
a. combining the Pseudo-ICE or the functional fragment with the pro-caspase-1 in the absence of a candidate compound under conditions that allow specific binding between the Pseudo-ICE or the functional fragment and the pro-capase-1 or the fragment thereof,
b. combining the Pseudo-ICE or the functional fragment with the pro-caspase-1 or the fragment thereof in the presence of the candidate compound under conditions of step (a); and
c. comparing the specific binding between the Pseudo-ICE or the functional fragment and the pro-caspase-1 or the fragment thereof of step (a) with that of step (b) to thereby determine whether the candidate compound is capable of decreasing the specific binding between the Pseudo-ICE or the functional fragment and the pro-caspase-1.

63. The method of claim 62 wherein the Pseudo-ICE has an amino acid sequence of SEQ ID NO: 1.

64. A process for manufacturing a compound that decreases the specific binding between a pro-caspase-1 or a fragment thereof that comprises a pro-domain of the pro-caspase-1 and a Pseudo-ICE having at least 80% amino acid identity to SEQ ID NO: 1 or a functional fragment of Pseudo-ICE capable of binding to the pro-caspase-1 or the fragment thereof, comprising:
a. carrying out the method of claim 48 to identify a compound that decreases the specific binding between the pro-caspase-1 or the fragment thereof and the Pseudo-ICE or the functional fragment of Pseudo-ICE;
b. derivatizing the compound; and
c. optionally repeating steps a and b.

65. The process of claim 64 wherein the Pseudo-ICE has an amino acid sequence of SEQ ID NO: 1.

66. A method for identifying a compound that disrupts the binding between a pro-caspase-1 or a fragment thereof that comprises a pro-domain of the pro-caspase-1 and a Pseudo-ICE having at least 80% amino acid identity to SEQ ID NO: 1 or a functional fragment of the Pseudo-ICE capable of binding to the pro-caspase-1 or the fragment thereof, comprising:
a. contacting a candidate compound with a binding complex comprising the Pseudo-ICE or the functional fragment and the pro-caspase-1; and
b. detecting the Pseudo-ICE, the functional fragment, the pro-caspase-1 or the fragment thereof that dissociates from the binding complex to thereby determine whether the candidate compound is capable of disrupting the binding between the pro-caspase-1 or the fragment thereof and the Pseudo-ICE or the functional fragment.

67. The method of claim 66 wherein the Pseudo-ICE has an amino acid sequence set forth in SEQ ID NO: 1.

68. The method of claim 66 wherein the Pseudo-ICE, the functional fragment, the pro-caspase-1 or the fragment thereof is covalently bound to a detectable moiety.

69. The method of claim 68 wherein the detectable moiety is a reporter molecule.

70. The method of claim 68 wherein the detectable moiety is radionuclide.

71. A process for manufacturing a compound that disrupts the specific binding between a pro-caspase-1 or a fragment thereof that comprises a pro-domain of the pro-caspase-1 and a Pseudo-ICE having at least 80% amino acid identity to SEQ ID NO: 1 or a functional fragment of Pseudo-ICE capable of binding to the pro-caspase-1 or the fragment thereof, comprising:
a. carrying out the method of claim 66 to identify a compound that disrupts the specific binding between the pro-caspase-1 or the fragment thereof and the Pseudo-ICE or the functional fragment of Pseudo-ICE;
b. derivatizing the compound; and
C. optionally repeating steps a and b.

72. The process of claim 71 wherein the Pseudo-ICE has an amino acid sequence of SEQ ID NO: 1.

73. A method for identifying a compound that increases the specific binding between a RICK and a Pseudo-ICE having at least 80% amino acid identity to SEQ ID NO: 1 or a functional fragment of Pseudo-ICE capable of binding to the RICK, comprising:
a. combining the Pseudo-ICE or the functional fragment with the RICK in the absence of a candidate compound under conditions that allow specific binding between the Pseudo-ICE or the functional fragment and the capase-1;
b. combining the Pseudo-ICE or the functional fragment with the RICK in the presence of the candidate compound under conditions of step (a);
c. comparing the specific binding between the Pseudo-ICE or the functional fragment and the RICK of step (a) with that of step (b) to thereby determine whether the candidate compound is capable of increasing the specific binding between the Pseudo-ICE or the functional fragment and the RICK.

74. The method of claim 73 wherein the Pseudo-ICE has an amino acid sequence of SEQ ID NO: 1.

75. A method for identifying a compound that decreases the specific binding between a RICK and a Pseudo-ICE having at least 80% amino acid identity to SEQ ID NO: 1 or a functional fragment of Pseudo-ICE capable of binding to a RICK, comprising:
a. combining the Pseudo-ICE or the functional fragment with the RICK in the absence of a candidate compound under conditions that allow specific binding between the Pseudo-ICE or the functional fragment and the RICK;
b. combining the Pseudo-ICE or the functional fragment with the RICK in the presence of the candidate compound under conditions of step (a); and
c. comparing the specific binding between the Pseudo-ICE or the functional fragment and the RICK of step (a) with that of step (b) to thereby determine whether the candidate compound is capable of decreasing the specific binding between the Pseudo-ICE or the functional fragment and the RICK.

76. The method of claim 75 wherein the Pseudo-ICE has an amino acid sequence of SEQ ID NO: 1.

77. A method for identifying a compound that disrupts the binding between a RICK and a Pseudo-ICE having at least 80% amino acid identity to SEQ ID NO: 1 or a functional fragment of the Pseudo-ICE capable of binding to the RICK, comprising:
a. contacting a candidate compound with a binding complex comprising the Pseudo-ICE or the functional fragment and the RICK; and
b. detecting the Pseudo-ICE, the functional fragment, or the RICK that dissociates from the binding complex to thereby determine whether the candidate compound is capable of disrupting the binding between the RICK and the Pseudo-ICE or the functional fragment.

78. The method of claim 77 wherein the Pseudo-ICE has an amino acid sequence set forth in SEQ ID NO: 1.

79. The method of claim 77 wherein the Pseudo-ICE, the functional fragment or the RICK is covalently bound to a detectable moiety.

80. The method of claim 79 wherein the detectable moiety is a reporter molecule.

81. The method of claim 79 wherein the detectable moiety is radionuclide.

82. A method of identifying inhibitors or enhancers of ICE-Like mediated inhibition of pro-caspase-1 activation, comprising:
a. contacting a cell transfected with an expression vector encoding ICE-Like having at least 80% amino acid identity to SEQ ID NO: 3 (ICE-Like amino acid sequence) or a functional fragment of the ICE-Like capable of inhibiting pro-caspase-1 activation with a candidate inhibitor or enhancer; and
b. detecting an increase or decrease in pro-caspase-1 activation in the presence of the candidate inhibitor or enhancer, wherein a decrease in pro-caspase-1 activation indicates the presence of an enhancer of ICE-Like mediated inhibition of pro-caspase-1 activation and an increase in pro-caspase-1 activation indicates the presence of an inhibitor of ICE-Like mediated inhibition of pro-caspase-1 activation.

83. The method of claim 82 wherein step (b) detects the level of pro-caspase-1 oligomerization.

84. The method of claim 82 wherein step (b) detects the level of caspase processing activity.

85. The method of claim 82 wherein step (b) detects the substrate cleavage activity of the caspase-1 processed from the pro-caspase-1.

86. The method of claim 82 wherein step (b) detects the level of IL-1β secretion induced by an inflammatory stimulus.

87. The method of claim 86 wherein the inflammatory stimulus is a lipopolysaccharide, an IFN-γ, or a TNF.

88. A method of identifying inhibitors or enhancers of ICE-Like mediated inhibition of the IL-1β secretion in response to an inflammatory stimulus, comprising:
a. contacting a cell transfected with an expression vector encoding ICE-Like having at least 80% amino acid identity to SEQ ID NO: 3 or a functional fragment of the ICE-Like capable of inhibiting IL-1β secretion with a candidate inhibitor or enhancer;
b. subjecting the transfected cell to an inflammatory stimulus; and
c. detecting an increase or decrease in the IL-1β secretion level induced by the inflammatory stimulus in the presence of the candidate inhibitor or enhancer, wherein a decrease in the induced IL-1β secretion indicates the presence of an enhancer of ICE-Like mediated inhibition of the induced IL-1β secretion and an increase in the induced IL-1β secretion indicates the presence of an inhibitor of ICE-Like mediated inhibition of the induced IL-1β secretion.

89. The method of claim 88 wherein the inflammatory stimulus is an IFN-γ, a TNF, or a lipopolysaccharide.

90. The method of claim 88 wherein the ICE-Like has an amino acid sequence set forth in SEQ ID NO: 3.

91. A method of identifying a polypeptide that specifically binds to an ICE-Like having at least 80% amino acid identity to SEQ ID NO: 3 or a functional fragment of the ICE-Like capable of specifically binding to pro-caspase-1 or inhibiting induced IL-1β secretion, comprising:
a. contacting a cDNA expression library with the ICE-Like or the functional fragment under conditions that permit the formation of a complex between the ICE-Like or the functional fragment thereof and a polypeptide encoded by a clone of the cDNA expression library; and
b. detecting the complex and the polypeptide in the complex.

92. The method of claim 91 wherein the ICE-Like has an amino acid sequence set forth in SEQ ID NO: 3.

93. The method of claim 91 wherein the ICE-Like or the functional fragment thereof is covalently bound to a detectable moiety.

94. The method of claim 93 wherein the detectable moiety is a reporter molecule.

95. The method of claim 93 wherein the detectable moiety is a radionuclide.

96. A nucleic acid molecule comprising a first nucleic acid sequence encoding an ICE-Like having at least 80% identity to SEQ ID NO: 3 or a functional fragment of the ICE-Like capable of specifically binding to pro-caspase-1 or inhibiting induced IL-1β secretion and a second nucleic acid sequence encoding the transcription activation domain or the DNA-binding domain of a transcription factor.

97. A method for identifying a polypeptide that specifically binds to an ICE-Like having at least 80% amino acid identity to SEQ ID NO: 3 or a functional fragment of the ICE-Like capable of specifically binding to pro-caspase-1 or inhibiting induced IL-1β secretion with a yeast two-hybrid screening system, comprising transforming a yeast cell with a vector comprising the nucleic acid molecule of claim 96.

98. A method for identifying a compound that increases the specific binding between a pro-caspase-1 or a fragment thereof that comprises a pro-domain of the pre-caspase-1 and an ICE-Like having at least 80% amino acid identity to SEQ ID NO: 3 or a functional fragment of ICE-Like capable of binding to the pro-caspase-1 or the fragment thereof, comprising:
a. combining the ICE-Like or the functional fragment with the pro-caspase-1 or the fragment thereof in the absence of a candidate compound under conditions that allow specific binding between the ICE-Like or the functional fragment and the capase-1 or the fragment thereof;
b. combining the ICE-Like or the functional fragment with the pro-caspase-1 or the fragment thereof in the presence of the candidate compound under conditions of step (a); and
c. comparing the specific binding between the ICE-Like or the functional fragment and the pro-caspase-1 or the fragment thereof of step (a) with that of step (b) to thereby determine whether the candidate compound is capable of increasing the specific binding between the ICE-Like or the functional fragment and the pro-caspase-1.

99. The method of claim 98 wherein the ICE-Like has an amino acid sequence of SEQ ID NO: 3.

100. A process for manufacturing a compound that increases the specific binding between a pro-caspase-1 or a fragment thereof that comprises a pro-domain of the pro-caspase-1 and an ICE-Like having at least 80% amino acid identity to SEQ ID NO: 3 or a functional fragment of ICE-Like capable of binding to the pro-caspase-1 or the fragment thereof, comprising:
a. carrying out the method of claim 98 to identify a compound that increases the specific binding between the pro-caspase-1 or the fragment thereof and the ICE-Like or the functional fragment;
b. derivatizing the compound; and
c. optionally repeating steps a and b.

101. The process of claim 100 wherein the ICE-Like has an amino acid sequence of SEQ ID NO: 3.

102. A method for identifying a compound that decreases the specific binding between a pro-caspase-1 and an ICE-Like having at least 80% amino acid identity to SEQ ID NO: 3 or a fumctional fragment of ICE-Like capable of binding to a pro-caspase-1, comprising:
a. combining the ICE-Like or the functional fragment with the pro-caspase-1 in the absence of a candidate compound under conditions that allow specific binding between the ICE-Like or the functional fragment and the capase-1;
b. combining the ICE-Like or the functional fragment with the pro-caspase-1 in the presence of the candidate compound under conditions of step (a);
c. comparing the specific binding between the ICE-Like or the functional fragment and the pro-caspase-1 of step (a) with that of step (b) to thereby determine whether the candidate compound is capable of decreasing the specific binding between the ICE-Like or the functional fragment and the pro-caspase-1.

103. The method of claim 102 wherein the ICE-Like has an amino acid sequence of SEQ ID NO: 3.

104. A process for manufacturing a compound that decreases the specific binding between a pro-caspase-1 and an ICE-Like having at least 80% amino acid identity to SEQ ID NO: 3 or a functional fragment of ICE-Like capable of binding to the pro-caspase-1, comprising:
a. carrying out the method of claim 102 to identify a compound that decreases the specific binding between the pro-caspase-1 and the ICE-Like or the functional fragment;
b. derivatizing the compound; and
c. optionally repeating steps a and b.

105. The process of claim 102 wherein the ICE-Like has an amino acid sequence of SEQ ID NO: 3.

106. A method for identifying a compound that disrupts the binding between a pro-caspase-1 or a fragment thereof that comprises a pro-domain of the pro-caspase-1 and an ICE-Like having at least 80% amino acid identity to SEQ ID NO: 3 or a functional fragment of the ICE-Like capable of binding to the pro-caspase-1 or the fragment thereof, comprising:
a. contacting a candidate compound with a binding complex comprising the ICE-Like or the functional fragment and the pro-caspase-1 or the fragment thereof; and
b. detecting the ICE-Like, the functional fragment, or the pro-caspase-1 or the fragment thereof that dissociates from the binding complex to thereby determine whether the candidate compound is capable of disrupting the binding between the pro-caspase-1 or the fragment thereof and the ICE-Like or the functional fragment.

107. The method of claim 106 wherein the ICE-Like has an amino acid sequence set forth in SEQ ID NO: 1.

108. The method of claim 106 wherein the ICE-Like, the functional fragment, the pro-caspase-1 or the fragment thereof is covalently bound to a detectable moiety.

109. The method of claim 108 wherein the detectable moiety is a reporter molecule.

110. The method of claim 108 wherein the detectable moiety is radionuclide.

111. A process for manufacturing a compound that disrupts the specific binding between a pro-caspase-1 or a fragment thereof that comprises a pro-domain of the pro-caspase-1 and an ICE-Like having at least 80% amino acid identity to SEQ ID NO: 3 or a functional fragment of ICE-Like capable of binding to the pro-caspase-1 or the fragment thereof, comprising:
a. carrying out the method of claim 107 to identify a compound that disrupts the specific binding between the pro-caspase-1 or the fragment thereof and the ICE-Like or the functional fragment;
b. derivatizing the compound; and
c. optionally repeating steps a and b.

112. The process of claim 111 wherein the ICE-Like has an amino acid sequence of SEQ ID NO: 3.


SEQUENCE LISTING (8 sequences)

SEQ ID NO: 1 - protein, 97 aa, Homo sapiens
MADKVLKEKRKLFIHSMGEGTINGLLDELLQTRVLNQEEMEKVKRENATVMDKTRALIDSVIPKGAQACQICITYICEEDSYLAETLGLSAGPIPGN

SEQ ID NO: 2 - DNA, 294 bp, Homo sapiens
atggccgacaaggtcctgaaggagaagagaaagctgtttatccattccatgggtgaaggtacaataaatggcttactggatgaattattacagacaagggtgctgaaccaggaagagatggagaaagtaaaacgtgaaaatgctacagttatggataagacccgagctttgattgactccgttattccgaaaggggcacaggcatgccaaatttgcatcacatacatttgtgaagaagacagttacctggcagagacgctgggactctcagcaggtccgatacctggaaattag

SEQ ID NO: 3 - protein, 90 aa, Homo sapiens
MADQLLRKKRRIFIHSVGAGTINALLDCLLEDEVISQEDMNKVRDENDTVMDKARVLIDLVTGKGPKSCCKFIKHLCEEDPQLASKMGLH

SEQ ID NO: 4 - DNA, 273 bp, Homo sapiens
atggctgaccaactcttgcgtaaaaagagaagaatttttatccattcagtgggtgcaggcacaataaatgccttgctggattgcctattagaggatgaagttattagccaggaagacatgaacaaagtgagagatgaaaatgacactgtcatggataaggctcgagtcttgattgaccttgttactggaaaaggacccaagtcttgctgcaaatttatcaagcatctctgtgaagaagaccctcaacttgcctcaaagatgggtttgcactaa

SEQ ID NO: 5 - protein, 103 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQD

SEQ ID NO: 6 - DNA, 429 bp, Homo sapiens
atggccgacaaggtcctgaaggagaagagaaagctgtttatccgttccatgggtgaaggtacaataaatggcttactggatgaattattacagacaagggtgctgaacaaggaagagatggagaaagtaaaacgtgaaaatgctacagttatggataagacccgagctttgattgactccgttattccgaaaggggcacaggcatgccaaatttgcatcacatacatttgtgaagaagacagttacctggcagggacgctgggactctcagcagatcaaacatctggaaattaccttaatatgcaagactctcaaggagtactttcttcctttccagctcctcaggcagtgcaggacaacccagctatgcccacatcctcaggctcagaagggaatgtcaagctttgctccctagaagaagctcaaagg

SEQ ID NO: 7 - DNA, 430 bp, Homo sapiens
atggccgacaaggtcctgaaggagaagagaaagctgtttatccattccatgggtgaaggtacaataaatggcttactggatgaattattacagacaagggtgctgaaccaggaagagatggagaaagtaaaacgtgaaaatgctacagttatggataagacccgagctttgattgactccgttattccgaaaggggcacaggcatgccaaatttgcatcacatacatttgtgaagaagacagttacctggcagagacgctgggactctcagcaggtccgatacctggaaattagcttagtacacaagactcccaattactattttcttccttcccagctcttcaggcagtgcgaggacaacccagctatgcccacatgctcaagcccagaaggcagaatcaagctttgctttctagaagacgctcaaggg

SEQ ID NO: 8 - DNA, 52 bp, Homo sapiens
atggccggtgagtaaacagacaaggcagcaggtaaggttataggtccgatag